SEQUENCE LISTING (21 sequences)

SEQ ID NO: 1 - DNA, 20 bp, Artificial Sequence
  Description of Artificial Sequence Synthetic oligonucleotide
ctgccactgcctacttgtgt

SEQ ID NO: 2 - DNA, 20 bp, Artificial Sequence
  Description of Artificial Sequence Synthetic oligonucleotide
gatggatgtgacctcattgc

SEQ ID NO: 3 - DNA, 20 bp, Artificial Sequence
  Description of Artificial Sequence Synthetic oligonucleotide
ctgctgctacagaggcagtg

SEQ ID NO: 4 - DNA, 21 bp, Artificial Sequence
  Description of Artificial Sequence Synthetic oligonucleotide
gacaaaacagagcgtcagcga

SEQ ID NO: 5 - DNA, 21 bp, Artificial Sequence
  Description of Artificial Sequence Synthetic oligonucleotide
tcacgccaccgagactaaata

SEQ ID NO: 6 - DNA, 21 bp, Artificial Sequence
  Description of Artificial Sequence Synthetic oligonucleotide
tgcagtagggtgtcatgaagg

SEQ ID NO: 7 - DNA, 22 bp, Artificial Sequence
  Description of Artificial Sequence Synthetic oligonucleotide
catcttgccaggaatcaacact

SEQ ID NO: 8 - DNA, 20 bp, Artificial Sequence
  Description of Artificial Sequence Synthetic oligonucleotide
accagggacaagcacaggta

SEQ ID NO: 9 - DNA, 23 bp, Artificial Sequence
  Description of Artificial Sequence Synthetic oligonucleotide
caaagccagagtccttcagagag

SEQ ID NO: 10 - DNA, 23 bp, Artificial Sequence
  Description of Artificial Sequence Synthetic oligonucleotide
ttagccactccttctgtgactcc

SEQ ID NO: 11 - DNA, 21 bp, Artificial Sequence
  Description of Artificial Sequence Synthetic oligonucleotide
cagcctcttctcattcctgct

SEQ ID NO: 12 - DNA, 21 bp, Artificial Sequence
  Description of Artificial Sequence Synthetic oligonucleotide
gccatttgggaacttctcatc

SEQ ID NO: 13 - DNA, 23 bp, Artificial Sequence
  Description of Artificial Sequence Synthetic oligonucleotide
ttgattaagtccctgccctttgt

SEQ ID NO: 14 - DNA, 19 bp, Artificial Sequence
  Description of Artificial Sequence Synthetic oligonucleotide
cgatccgagggcctcacta

SEQ ID NO: 15 - protein, 524 aa, Unknown
  Description of Unknown TNAP sequence
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

SEQ ID NO: 16 - DNA, 584 bp, Artificial Sequence
  Description of Artificial Sequence Synthetic polynucleotide
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 17 - protein, 10 aa, Artificial Sequence
  Description of Artificial Sequence Synthetic peptide
DDDDDDDDDD

SEQ ID NO: 18 - protein, 3 aa, Artificial Sequence
  Description of Artificial Sequence Synthetic peptide
DSS

SEQ ID NO: 19 - protein, 8 aa, Artificial Sequence
  Description of Artificial Sequence Synthetic peptide
KRRTPVRE

SEQ ID NO: 20 - protein, 8 aa, Artificial Sequence
  Description of Artificial Sequence Synthetic peptide
KNFQSRSH

SEQ ID NO: 21 - protein, 8 aa, Artificial Sequence
  Description of Artificial Sequence Synthetic peptide
KTYASMQW

(19) United States
(12) Patent Application Publication
(10) Pub. No.: US 2025/0034534 A1
(43) Pub. Date: Jan. 30, 2025

(54) METHODS AND COMPOSITIONS FOR TREATING HYPOPHOSPHATASIA

(71) Applicants: Koichi MIYAKE, Tokyo (JP); Sanford Burnham Prebys Medical Discovery Institute, La Jolla, CA (US)

(72) Inventors: Koichi MIYAKE, Tokyo (JP); Jose Luis MILLAN, Valley Center, CA (US)

(21) Appl. No.: 18/562,705

(22) PCT Filed: May 25, 2022
(86) PCT No.: PCT/US2022/030928

§ 371 Date: Nov. 20, 2023

Related U.S. Application Data
(60) Provisional application No. 63/193,366, filed on May 26, 2021.

(51) Int. Cl. C12N 9/16; A61K 38/46; A61K 48/00; A61P 19/08; C12N 15/86

(52) U.S. Cl. CPC C12N 9/16; A61K 48/005; A61P 19/08; C12N 15/86; A61K 38/465; C07K 2319/33; C12N 2750/14143; C12Y 301/03001

(57) ABSTRACT

Described herein are compositions and methods useful for treating a soft bone disease, or for treating hypophosphatasia comprising administering a viral vector comprising a mineral-targeted alkaline phosphatase under the control of a tissue non-specific promotor to the subject in an intramuscular injection to a muscle, wherein administering the viral vector treats the soft bone disease. The compositions disclosed herein are suitable for administration to a subject.

### CROSS-REFERENCE

This application claims the benefit of U.S. Provisional Patent Application No. 63/193,366, filed May 26, 2021, which application is entirely incorporated herein by reference.

### STATEMENT AS TO FEDERALLY SPONSORED RESEARCH

This invention was made with government support under Grant No. DE012889 awarded by the National Institutes of Health. The government has certain rights in the invention.

### BACKGROUND

Hypophosphatasia (HPP) is an inborn error-of-metabolism caused by loss-of-function mutations in the ALPL gene, which encodes tissue-nonspecific alkaline phosphatase (TNAP). TNAP is expressed in bones, teeth, liver, and kidney; its deficiency leads to mineralization defects caused by the accumulation of extracellular inorganic pyrophosphate (PPᵢ), one of the major substrates of TNAP and a potent inhibitor of hydroxy apatite crystal formation and propagation. Murine studies have demonstrated that mineralizing skeletal and dental cells, including osteoblasts, chondrocytes, ameloblasts, odontoblasts, and cementoblasts, express TNAP.

### SUMMARY

In certain aspects, disclosed herein is a method of treating a subject with a soft bone disease comprising: administering a viral vector comprising a mineral-targeted alkaline phosphatase under the control of a tissue non-specific promotor to the subject in an intramuscular injection to a muscle, wherein administering the viral vector treats the soft bone disease. In some embodiments, the tissue non-specific promotor comprises a CAG promotor. In some embodiments, the viral vector comprises an adeno-associated vector. In some embodiments, the viral vector comprises an adeno-associated virus type 8 (AAV8) vector. In some embodiments, the mineral-targeted alkaline phosphatase comprises tissue non-specific alkaline phosphatase (TNAP). In some embodiments, the mineral-targeted alkaline phosphatase comprises tissue non-specific alkaline phosphatase (TNAP) and a bone targeting sequence linked to the C-terminus of TNAP. In some embodiments, the bone targeting sequence is a deca-aspartate (D₁₀) sequence. In some embodiments, the soft bone disease is hypophosphatasia (HPP). In some embodiments, the soft bone disease is hypophosphatasia (HPP), and wherein the hypophosphatasia is pediatric hypophosphatasia or infantile hypophosphatasia. In some embodiments, the soft bone disease is hypophosphatasia (HPP), and wherein the hypophosphatasia is late-onset hypophosphatasia. In some embodiments, the soft bone disease is caused by PHOSPHO1 deficiency. In some embodiments, the subject is a human. In some embodiments, the subject is a mouse. In some embodiments, the mouse comprises a TNAP knockout mouse. In some embodiments, administering the viral vector results in increased plasma alkaline phosphatase (ALP) activity for at least two months. In some embodiments, following administering the viral vector, the viral vector is not detected in the brain. In some embodiments, following administering the viral vector, the viral vector is not detected in the gonads. In some embodiments, following administering the viral vector, the viral vector does not result in oncogenic effect in the subject. In some embodiments, the viral vector does not diffuse from the muscle.

In certain aspects, disclosed herein is a composition comprising a viral vector comprising a mineral-targeted alkaline phosphatase under the control of a tissue non-specific promotor. In certain aspects, disclosed herein is a composition for treating a soft bone disease, comprising a viral vector comprising a mineral-targeted alkaline phosphatase under the control of a tissue non-specific promotor, wherein the composition treats the soft bone disease and wherein the composition results in plasma ALP activity higher than in a subject not receiving the composition for at least eighteen months. In certain aspects, disclosed herein is a composition for treating a soft bone disease, comprising a viral vector comprising a mineral-targeted alkaline phosphatase under the control of a tissue non-specific promotor, wherein the composition treats the soft bone disease and wherein the viral vector does not diffuse from a target location. In some embodiments, the tissue non-specific promotor comprises a CAG promotor. In some embodiments, the viral vector comprises an adenoviral-associated virus. In some embodiments, the adenoviral-associated virus comprises an adeno-associated virus type 8 (AAV8) vector. In some embodiments, the mineral-targeted alkaline phosphatase comprises tissue non-specific alkaline phosphatase (TNAP). In some embodiments, the mineral-targeted alkaline phosphatase comprises a sequence for bone targeting linked to the C-terminus of TNAP. In some embodiments, the bone targeting sequence is a deca-aspartate (D₁₀) sequence. In some embodiments, the soft bone disease is hypophosphatasia (HPP). In some embodiments, the hypophosphatasia is pediatric hypophosphatasia or infantile hypophosphatasia. In some embodiments, the hypophosphatasia is late-onset hypophosphatasia. In some embodiments, the soft bone disease is caused by PHOSPHO1 deficiency. In some embodiments, disclosed herein is a method of treating a soft bone disease, the method comprising administering the composition described herein to the subject in an intramuscular injection to a muscle.

In certain aspects, disclosed herein is a method of treating a subject with a dental disorder comprising: administering a viral vector comprising a mineral-targeted alkaline phosphatase under the control of a tissue non-specific promotor to the subject in an intramuscular injection, wherein administering the viral vector treats the dental disorder. In some embodiments, the tissue non-specific promotor comprises a CAG promotor. In some embodiments, the viral vector comprises an adenoviral-associated virus. In some embodiments, the viral vector comprises an adeno-associated virus type 8 (AAV8) vector. In some embodiments, the mineral-targeted alkaline phosphatase comprises tissue non-specific alkaline phosphatase (TNAP). In some embodiments, the mineral-targeted alkaline phosphatase further comprises a sequence for bone targeting linked to the C-terminus of TNAP. In some embodiments, the bone targeting sequence is a deca-aspartate (D₁₀) sequence. In some embodiments, the dental disorder comprises at least one of a dentoalveolar disorder, teeth hypomineralization, and a periodontal disorder

### INCORPORATION BY REFERENCE

All publications, patents, and patent applications mentioned in this specification are incorporated by reference herein to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent application contains at least one drawing executed in color. Copies of this patent or patent application with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1G depict that AAV8-TNAP-D₁₀ improves survival and altered PPᵢ metabolism in AAV8-TNAP-D₁₀-treated Alpl^{-/-} mice. FIG. 1A depicts average body weight of AAV8-TNAP-D₁₀-treated male Alpl^{-/-} mice is comparable to WT littermates, though female Alpl^{-/-} mice have lower body weight than WT. FIG. 1B depicts that AAV8-TNAP-D₁₀-treated Alpl^{-/-} and AAV8-TNAP-D₁₀-treated Alpl^{-/-} WT mice show a significant increase in serum ALP activity compared to untreated WT mice. FIG. 1C depicts that plasma PPᵢ levels of AAV8-TNAP-D₁₀-treated Alpl^{-/-} mice are significantly lower than those of WT controls. FIG. 1D depicts that urine PPᵢ concentrations of AAV8-TNAP-D₁₀-treated Alpl^{-/-} mice remain significantly higher than both treated and untreated WT mice (70 dpn). FIG. 1E depicts that urine PPᵢ concentrations of untreated Alpl^{-/-} and heterozygote (Alpl^{+/-}) mice are higher than those of WT mice (10 dpn). FIG. 1F depicts that qPCR of kidney RNA shows reduced Alpl expression in Alpl^{-/-} mice, but no significant differences in other genes related to PP₁ metabolism (Ank, Enpp1, and Abcc6) or genes related to inflammation (Il6 and Tnf). FIG. 1G depicts that histochemical staining of the kidney reveals no ALP activity in the proximal tubules of AAV8-TNAP-D₁₀-treated Alpl^{-/-} mice (upper panel). Light microscopy does not reveal any apparent structural changes in the H&E-stained glomeruli or renal tubules of AAV8-TNAP-D₁₀-treated Alpl^{-/-} mice (lower panel).

FIGS. 2A-2D depict improved radiographical findings in AAV8-TNAP-D₁₀-treated Alpl^{-/-} mice. FIG. 2A depicts radiographs of the whole skeleton of a female WT littermate control and a female Alpl^{-/-} mouse treated with AAV8-TNAP-D₁₀. FIG. 2B depicts that AAV8-TNAP-D₁₀-treated Alpl^{-/-} mice showed no signs of rickets, such as bowing and flaring of the metaphysis, and craniosynostosis. FIG. 2C depicts that AAV8-TNAP-D₁₀-treated female Alpl^{-/-} mice have shorter limb lengths compared to their WT littermates. FIG. 2D depicts that the nose length, cranial length, and cranial width are not significantly different among the groups. Unpaired t-test with Welch's correction was performed to compare the differences between WT and treated Alpl^{-/-} male mice. One-way ANOVA followed by Turkey's multiple comparisons test was performed to compare the differences among WT, treated Alpl^{-/-}, and treated WT female mice. Significance is shown in the graph as *P<0.05, **P<0.01, ***P<0.001.

FIG. 3A-3C depict partial normalization of bone microstructure in AAV8-TNAP-D₁₀-treated Alpl−/− mice. FIG. 3A depicts 2D and 3D microCT images showing femurs from treated Alpl^{-/-} mice compared with WT controls. Treated Alpl^{-/-} females show shorter femurs compared with WT controls. Red arrows point to abnormal articular surfaces of medial and distal femurs in the treated Alpl^{-/-} mice. FIG. 3B depicts quantification of trabecular bone parameters from 50 slices proximal to the growth plate of distal femurs. FIG. 3C depicts quantification of cortical bone parameters from 50 slices from femoral midshaft. Statistical analysis performed by the one-way ANOVA followed by Tukey's multiple comparison test where *P<0.05; **P<0.01.

FIG. 4A-4D depict improved histological findings in AAV8-TNAP-D₁₀-treated Alpl^{-/-} mice. FIG. 4A depicts histological analysis of femur of WT and the AAV8-TNAP-D₁₀-treated Alpl^{-/-} mice. Von Kossa/van Gieson staining of the femur bones and the lumbar spines show no measurable osteoid surface. FIG. 4B depicts that BV/TV values of the lumbar spine of AAV8-TNAP-D₁₀-treated Alpl^{-/-} mice are significantly lower than that of WT mice. (WT n=5; male n=2, female n=3, KO n=3; male n=1, female n=2). FIG. 4C depicts H&E and Safranin O staining of the decalcified tibial bones show an abnormal distribution of chondrocytes in the secondary ossification center in AAV8-TNAP-D₁₀-treated Alpl^{-/-} mice (black arrows). FIG. 4D depicts histochemical staining of the femur bones shows strong ALP activity in the hypertrophic zone of the epiphyseal growth plate, metaphysis, and diaphysis in WT mice, while only a subtle ALP activity is observed in the growth plate and diaphysis of AAV8-TNAP-D₁₀-treated Alpl^{-/-} mice.

FIG. 5A-5I depict AAV8-TNAP-D₁₀ prevents HPP-associated dentoalveolar defects in Alpl^{-/-} mice. FIG. 5A-5F depict that 3D and 2D micro-CT renderings of first molars (M1) and incisors (INC) exhibit normal tooth structures in AAV8-TNAP-D₁₀-treated Alpl^{-/-} mice similar to those in WT controls (70 dpn). FIG. 5G depicts that first molars show no significant differences in enamel density, dentin volume, or dentin density among the groups. Tooth enamel shows decreased volume in treated Alpl^{-/-} molars compared with WT controls. FIG. 5H depicts that continually erupting incisor teeth show no significant defects in the volume or density of either enamel or dentin among the groups. FIG. 5I depicts that AAV8-TNAP-D₁₀ significantly improves alveolar bone volume in treated Alpl^{-/-} vs. WT mice, but alveolar bone shows 4% less mineral density in treated Alpl^{-/-} vs. WT mice. Statistical analysis performed by the one-way ANOVA followed by Tukey's multiple comparison test where *P<0.05; **P<0.01; ***P<0.001; ****P<0.0001. EN=enamel; DE=dentin; AB=alveolar bone.

FIG. 6A-6G depict improved cementum and PDL attachment in AAV8-treated Alpl^{-/-} mice. FIG. 6A depicts that H&E staining shows no evident dental defects in treated Alpl^{-/-} mice compared with WT controls. Boxed regions are shown at higher magnification (left). FIG. 6B depicts that in situ hybridization with Alpl probe (left) confirms the absence of Alpl expression in treated Alpl^{-/-} mice. TNAP IHC (right) shows weak staining (brown) around treated Alpl^{-/-} alveolar bone (AB) and more robust staining in WT mice. FIG. 6C depicts that BSP IHC (left) shows evident staining (brown) in acellular cementum (AC, white arrow) and alveolar bone (AB) in treated Alpl^{-/-} and WT mice. OPN IHC (right) shows comparable staining (brown) in AC and AB in treated Alpl^{-/-} vs. WT mice. FIG. 6D depicts that ImageJ color map (left) shows improved cellularity (yellow symbols) within the PDL space of treated Alpl^{-/-} vs. WT mice, and (right) shows the width of AC (yellow lines) in treated Alpl^{-/-} vs. WT mice. FIG. 6E-6G depict quantification of the thickness of acellular cementum, mantle dentin (MD), and cellularity in the PDL space. Statistical analysis performed by the one-way ANOVA followed by Tukey's multiple comparison test where *P<0.05 and **P<0.01, ns: not significant.

FIG. 7A-7B depict size of Alpl^{-/-} and WT pups. FIG. 7A shows untreated Alpl^{-/-} mice were significantly smaller than WT mice at 10 dpn. FIG. 7B depicts that Alpl^{-/-} mice injected with AAV8-TNAP-D₁₀ within five dpn were not significantly smaller than WT mice at 15 dpn.

FIG. 8 depicts that histochemical staining of the liver reveals no ALP activity in the branches of hepatic artery of AAV8-TNAP-D₁₀-treated Alpl^{-/-} mice, which is apparent in WT mice (black arrow).

FIG. 9 depicts that Von Kossa staining shows no ectopic calcifications in the aorta, coronary arteries, brain, or kidney in WT and AAV8-TNAP-D₁₀-treated Alpl^{-/-} mice at 70 dpn.

FIG. 10 depicts that Safranin O staining of the decalcified tibias shows an abnormal distribution of chondrocytes in secondary ossification centers in AAV8-TNAP-D₁₀-treated Alpl^{-/-} mice (black arrows). Slides were scanned by Aperio AT2 system to capture images of the entire tibia. The WT and Alpl^{-/-} images shown in the upper panel were the same as those shown in FIG. 4C (observed with microscopy).

FIGS. 11A-11C depict hypomineralization and impaired periodontium in untreated Alpl^{-/-} teeth compared with WT mice. FIG. 11A depicts that H&E staining of the first mandibular molar showing hypomineralized molar roots (upper panel, red arrow). The lower panel shows hypoplasia of acellular cementum (*) and loss of periodontal attachment. FIG. 11B depicts an ImageJ color map of H&E-stained images showing abnormal PDL cells in Alpl^{-/-} mice vs. WT mice. FIG. 11C depicts a bar graph of cell counts in the boxed region showing a fewer PDL cells, although not significant, in Alpl^{-/-} mice compared with WT mice. Statistical analysis performed by the student t test, where ns: not significant.

FIGS. 12A-12L depict the biochemical analysis in serum/plasma from adult HPP mice and WT littermates under AAV8-TNAP-D₁₀ treatment or AAV8-GFP control, 60 days after injection. FIGS. 12A-12B depict body weight. FIGS. 12C-12D depict serum alkaline phosphatase activity. FIGS. 12E-12F depict plasma PPᵢ levels. FIGS. 12G-12H depict serum calcium concentrations. FIGS. 12I-12J depict serum phosphorus concentrations. FIGS. 12K-12L depict blood urea nitrogen (BUN) levels in serum. Statistical analysis was performed by one-way ANOVA followed by Tukey's multiple comparison test. *P<0.05. **P<0.01. ***P<0.001. ****P<0.0001.

FIGS. 13A-13J depict a biochemical analysis in serum/plasma from 2 months old females and males adult HPP mice and WT siblings prior to injection. FIGS. 13A-13B depict serum alkaline phosphatase activity. FIGS. 13C-13D depict plasma PPi levels. FIGS. 13E-13F depict serum calcium assay. FIGS. 13G-13H depict serum phosphorus concentration. FIGS. 13I-13J depict blood urea nitrogen (BUN) levels in serum. Statistical analysis was performed by Unpaired t-test. *P<0.05. **P<0.01. ****P<0.0001.

FIGS. 14A-14J depict a biochemical analysis in serum/plasma from Phospho1 KO mice under AAV8-TNAP-D₁₀ treatment or AAV8-GFP as control after 45 and 90 days of injection. WT littermates were treated with AAV8-TNAP-D₁₀ vector. FIGS. 14A-14B depict serum alkaline phosphatase activity. FIGS. 14C-14D depict plasma PPᵢ levels. FIGS. 14E-14F depicts serum calcium concentrations. FIGS. 14G-14H depict serum phosphorus concentrations. FIGS. 14I-14J depict blood urea nitrogen (BUN) levels in serum. Statistical analysis was performed by one-way ANOVA followed by Tukey's multiple comparison test. *P<0.05. **P<0.01. ***P<0.001. ****P<0.0001.

FIGS. 15A-15D depict the radiographical findings of females adult HPP mice bone phenotype. Radiographic images of whole skeletal tissue, with higher magnification of skull along with spine (2×), vertebra (2×) and hemi-mandibles (3×). FIGS. 15A-15B depict females WT treated with control AAV8-GFP and AAV8-TNAP-D₁₀. FIGS. 15C-15D depict females adultHPP AAV8-GFP or AAV8-TNAP-D₁₀ treated mice after 60 days of injection.

FIGS. 16A-16D depict the radiographical findings of males adult HPP mice bone phenotype. Radiographic images of whole skeletal tissue, with higher magnification of skull along with spine (2×), vertebra (2×) and hemi-mandibles (3×). FIGS. 16A-16B depicts males WT treated with vehicle AAV8-GFP and AAV8-TNAP-D₁₀. FIGS. 16C-16D depicts males adult HPP AAV8-GFP or AAV8-TNAP-D₁₀ treated mice after 60 days of injection.

FIGS. 17A-17B depict the radiographic findings and H&E staining of long bones in adult HPP mice bone phenotype. FIG. 17A depicts X-ray images from female and male HPP mice and WT littermate control treated with AAV8-TNAP-D₁₀ or AAV8-GFP after 60 days of injection. Impaired long bones from HPP mice with defects in the proximal and distal femur along with the patellar joint surface and, proximal tibia. TNAP treatment partially rescued the epiphyseal and metaphyseal bone regions (Highlighted in red). FIG. 17B depicts H&E staining showed the disorganization of the growth plate in AAV8-GFP treated HPP mice, and a substantial improvement of the bone morphologic parameters in AAV8-TNAP-D₁₀ treated HPP mice.

FIGS. 18A-18B depict micro-CT analysis of femur from adult HPP mice and WT littermates. FIG. 18A depicts 2D and 3D micro-CT images of femurs from female and male treated with AAV8-TNAP-D₁₀ or AAV8-GFP after 60 days of injection. FIG. 18B depicts the micro-CT analysis of bone parameters in the femurs.

FIGS. 19A-19B depict radiographical findings of females Phospho1 KO mice bone phenotype. Radiographic images of whole skeletal tissue, with higher magnification of skull along with spine (2×), head (4×), hemi-mandibles (3×), vertebra (2×) and long bones (2×). Females Phospho1 KO treated with control AAV8-GFP or AAV8-TNAP-D₁₀ after 90 days of injection.

FIGS. 20A-20B depict a radiographical findings of females Phospho1 KO mice bone phenotype. Radiographic images of whole skeletal tissue, with higher magnification of skull along with spine (2×), head (4×), hemi-mandibles (3×), vertebra (2×) and long bones (2×). Males Phospho1 KO treated with control AAV8-GFP or AAV8-TNAP-D₁₀ after 90 days of injection.

FIG. 21A depicts representative X-ray images of the spine from female and male 90-days old Phospho1 KO mice. A single dose of AAV8-TNAP-D₁₀ or AAV8-GFP, as control (3×10¹¹ vg/body) was intramuscularly administered on 3 days old mice. The control AAV8-GFP treated Phospho1 KO mice showed scoliosis. Gene therapy using the AAV8-TNAP-D₁₀ vector corrected the scoliosis deformity of the spine. FIG. 21B depicts H&E staining showing a different tissue organization of vertebrae from mice treated with AAV8-GFP or the vector encoding TNAP, revealing increased trabecular bone and less bone marrow area in AAV8-TNAP-D₁₀ treated mice.

FIGS. 22A-22C depict micro-CT analysis of tibiae from Phospho1 KO mice and WT littermates. FIG. 22A depicts 2D micro-CT images of tibiae from females and males treated with AAV8-TNAP-D₁₀ or AAV8-GFP after 90 days of injection. FIGS. 22B-22C depict quantification of bone parameters.

FIGS. 23A-23B depict Alizarin red staining of soft organs from adult HPP and WT mice. Female and Male treated with AAV8-GFP or AAV8-TNAP-D₁₀. No evidence of ectopic calcifications was found after 60 days of vector encoding TNAP injection. Upper panels: kidney. Mid panels: Heart. Lower panels: Aorta (20×mag.).

FIGS. 24A-24B depict ectopic calcification on soft organs from adult females HPP and WT mice under CKD diet. FIG. 24A depicts Alizarin red staining that was performed to show the ectopic calcification in soft organs and vasculature in late-onset HPP mouse model and WT littermates as control. FIG. 23B depicts histological sections of kidney, heart, and aorta for the following experimental groups WT mice treated with control AAV8-GFP or AAV8-TNAP-D₁₀, and adult HPP injected mice with AAV8-GFP or AAV8-TNAP-D₁₀. Kidney: upper panels—996 μM, lower panels—100 μM mag; Heart: upper panels—996 μM, lower panels—50 μM mag; aorta: upper panels—100 μM, lower panels—50 μM mag. FIG. 24B depicts Alizarin red S quantification.

FIG. 25A-25C depict micro-CT analysis of dentoalveolar complex from adult HPP mice and WT littermates. FIG. 25A depicts 2D and 3D micro-CT images of first molar and incisor from AAV8-TNAP-D₁₀ or AAV8-GFP treated mice after 60 days of injection. FIG. 25B depicts quantification of enamel, dentin, alveolar bone, and pulp parameters. FIG. 25C depicts H&E staining of tissue organization of teeth. No obvious difference in tissue organization, periodontal attachments, or acellular cementum between AAV8-TNAP-D₁₀ or AAV8-GFP treated mice. M1: First molar. EN: Enamel. DE: Dentin. AB: Alveolar bone.

FIGS. 26A-26D depict micro-CT analysis of dentoalveolar complex from Phospho1 KO and WT littermates. mice. FIGS. 26A-26C depict 2D and 3D micro-CT images of first molar and incisor from AAV8-TNAP-D₁₀ or AAV8-GFP treated mice after 90 days of injection. FIG. 26D depicts a quantification of enamel, dentin, alveolar bone, and cellular cementum parameters. No obvious difference in tissue organization, periodontal attachments, except for higher dentin and acellular cementum volumes from AAV8-TNAP-D₁₀ treated WT mice when compared to Phospho1 KO mice treated with AAV8-TNAP-D₁₀ and AAV8-GFP.

### DETAILED DESCRIPTION

Hypophosphatasia (HPP) is caused by loss-of-function mutations in the ALPL gene that encodes tissue-nonspecific alkaline phosphatase (TNAP), whose deficiency results in the accumulation of extracellular inorganic pyrophosphate (PPᵢ), a potent mineralization inhibitor. Skeletal and dental hypomineralization characterizes HPP, with disease severity varying from life-threatening perinatal or infantile forms to milder forms that manifest in adulthood or only affect the dentition.

Asfotase alfa is a recombinant fusion protein comprising the TNAP ectodomain, a human IgG1 Fc domain for one-step purification, and a terminal deca-aspartate (D₁₀) motif for mineral-targeting. In a murine model for infantile HPP, TNAP knockout (Akp2−/− or Alpl−/−) mice, treatment with daily subcutaneous injections of asfotase alfa preserved life span, improved skeletal phenotypes, and prevented epileptic seizures and dental defects. In humans, subcutaneous injections of asfotase alfa, three to seven times a week, in children or adults with HPP has demonstrated substantial and sustained efficacy with a good safety profile. Asfotase alfa saved lives of subjects with severe neonatal and infantile HPP and improved bone mineralization, motor function, and quality of life in subjects with adult HPP. At the same time, the patient burden of multiple injections per week to maintain the efficacy of asfotase alfa and the associated medical cost have prompted preclinical studies of alternative strategies for treating HPP.

A human chimeric recombinant alkaline phosphatase, ChimAP, and several forms of virus vectors expressing TNAP-D₁₀ prolongs life, prevent seizures, and improve the skeletal phenotype of Alpl^{-/-} mice. A single intravenous injection of a lentiviral or adeno-associated virus type 8 (AAV8) vector encoding TNAP-D₁₀ leads to sustained correction of the skeletal phenotype of Alpl^{-/-} mice, but the consequential wide distribution of vector genome to the whole body raised concern about possible transduction into germ cells.

Previous approaches to correct HPP-associated mineralization defects have met with mixed success or have had inherent limitations or drawbacks. ERT using asfotase alfa was shown to be very effective at correcting skeletal and dentoalveolar defects in Alpl^{-/-} mice, with these translational studies leading to its approval in 2015 for treatment of patients with perinatal/infantile- and juvenile-onset HPP, except in Japan where asfotase alfa is approved for all ages. However, asfotase alfa ERT has a half-life of 2.28 days, requires multiple injections per week, is associated with injection site reactions, and is expensive, prompting additional preclinical studies of alternative strategies for HPP treatment. Daily subcutaneous injection of a soluble, non-mineral-targeting, recombinant chimeric alkaline phosphatase (ChimAP) prevented seizures, increased survival, but only partially improved the skeletal and dentoalveolar phenotypes in Alpl^{-/-} mice. Studies using several different types of viral vectors with different modes of administration have been reported. Single intravenous injection of either lentiviral vector containing TNAP-D₁₀ (HIV-TNAP-D₁₀) or AAV8-TNAP-D₁₀ resulted in sustained elevation of circulatory TNAP and phenotypic correction in Alpl^{-/-} mice, but the viral sequence was detected in soft tissues, including the liver, lung, and heart, raising a safety concern for oncogenicity of the integrated lentiviral vector in these tissues. At the same time, intramuscular injection of scAAV8-MCK-TNAP-D₁₀, a muscle-directed gene therapy consisted of a self-complementary type 8 AAV (scAAV8) vector and the muscle creatine kinase (MCK) promoter to limit the virus distribution, improved the skeletal phonotype in the Alpl^{-/-} mice, but with limited elongation of the long bone and remaining hypomineralization. When compared to these previous methods, the approach described herein using a single intramuscular injection of AAV8-TNAP-D₁₀, combines a more practical gene therapy, potentially improved safety profile due to limited tissue distribution, and yet remains highly effective at correcting mineralization defects.

Therapies for HPP other than asfotase alfa have been tested in a clinical setting without rigorous preclinical and translational studies. In a cross-sectional study that enrolled 51 patients with childhood and adult onset HPP, two out of four patients who were treated with teriparatide (parathyroid hormone 1-34) showed clinical and radiological improvement. In a phase IIA open-label study targeting eight adult patients with HPP, monoclonal antisclerostin antibody (BPS804) treatment resulted in increases in bone formation markers and bone mineral density. However, these anabolic agents have not been approved for HPP and an effective medical therapy for adult-onset HPP patients is needed. At the same time, a novel therapy with fewer injections may further benefit patients with childhood-onset HPP as well.

Concerns for AAV8-TNAP-D₁₀ administration in human patients include continuously elevated circulatory ALP activity and its potential effects on PPᵢ metabolism and development or aggravation of ectopic calcification. As described herein, a single intramuscular injection of AAV8-TNAP-D₁₀ resulted in extremely high circulatory ALP activity, more than 500 times higher than that of WT mice. Extracellular PPᵢ is known as a central regulator of biomineralization and is critical for controlling inappropriate soft tissue calcification in the body. Hydrolysis of PPᵢ by TNAP-D₁₀ may result in over-suppression of extracellular PPᵢ in soft tissues. However, AAV8-TNAP-D₁₀-treated Alpl^{-/-} mice, with suppressed plasma PPᵢ concentrations, did not develop any soft tissue calcifications during the observational window of 70 dpn. This contrasts with previous results from genetically modified mouse models with targeted overexpression of TNAP in the vasculature. TNAP overexpression in the smooth muscle cells using Tagln-Cre resulted in massive arterial calcifications in the ascending and descending aorta, carotid, and subclavian arteries. TNAP overexpression in endothelial cells using Tie2-Cre resulted in the partial calcification in the arteries of the heart, kidney, mesentery, pancreas, and spleen. These mouse models showed 20-30 times higher circulatory ALP activity while plasma PPᵢ concentrations were similar to those of WT. From these results, one can assume that neither circulatory ALP activities nor circulatory PPᵢ concentrations correlate with local PPᵢ concentrations, which cannot be measured in vivo but presumably are low in target tissues with high TNAP expression. Additionally, the elevated urine PPᵢ concentrations in TNAP-D₁₀-treated Alpl^{-/-} mice indicate that urine PPᵢ metabolism is mostly dependent on TNAP expressed in the luminal surface of kidney proximal tubules, and independent of serum ALP activity and circulatory PPᵢ concentrations. The qPCR analysis of the kidneys of treated mice indicated that genes for primary regulators of systemic PPᵢ metabolism, including Ank, Enpp1, and Abcc6, were not affected by treatment.

Described herein is the efficacy of a single intramuscular administration of adeno-associated virus 8 (AAV8) encoding TNAP-D₁₀ for increasing lifespan and improving the skeletal and dentoalveolar phenotypes in TNAP knockout (Alpl^{-/-}) mice, a murine model for severe infantile HPP. Alpl^{-/-} mice received 3×10¹¹ vector genomes/body of AAV8-TNAP-D₁₀ within 5 days postnatal (dpn). AAV8-TNAP-D₁₀ elevated serum ALP activity and suppressed plasma PPᵢ. Treatment extended lifespan of Alpl^{-/-} mice and no ectopic calcifications were observed in the kidneys, aorta, coronary arteries, or brain in the 70 dpn observational window. Treated Alpl^{-/-} mice did not show signs of rickets, including bowing of long bones, enlargement of epiphyses, or fractures. Bone microstructure of treated Alpl^{-/-} mice was similar to wild-type (WT), with a few persistent small cortical and trabecular defects. Histology showed no measurable osteoid accumulation, but reduced bone volume fraction in treated Alpl^{-/-} mice versus controls. Treated Alpl^{-/-} mice featured normal molar and incisor dentoalveolar tissues, with the exceptions of slightly reduced molar enamel and alveolar bone density. Histology showed the presence of cementum and normal periodontal ligament attachment. These results support gene therapy as a potential alternative to ERT for the treatment of HPP.

### Methods

Disclosed herein, in some embodiments, are methods useful for treating a subject in need thereof comprising administering a composition described herein. In some embodiments, the composition described herein comprises a viral vector described herein. In some embodiments, the disease is a soft bone disease, a dental disorder, or a combination thereof. In some embodiments, the method comprises administering a viral vector comprising a mineral-targeted alkaline phosphatase under the control of a tissue non-specific promotor to the subject in an intramuscular injection to a muscle.

In certain aspects, described herein is a method of administering a composition described herein to a subject in need thereof. In some embodiments, the methods improve at least one of the symptoms selected from the list consisting of life span, skeletal abnormalities, seizures, dental defects, bone mineralization, motor function, and quality of life. In some embodiments, the symptom is improved compared to a subject who has not received the treatment. In some embodiments, the symptom is improved compared to the subject prior to receiving the treatment.

In some embodiments, described herein is a method of increasing life span in a subject in need thereof, the method comprising administering the compositions described herein to the subject. In some embodiments, described herein is a method of reducing skeletal abnormalities in a subject in need thereof, the method comprising administering the compositions described herein to the subject. In some embodiments, described herein is a method of reducing seizures in a subject in need thereof, the method comprising administering the compositions described herein to the subject. In some embodiments, described herein is a method of reducing dental defects in a subject in need thereof, the method comprising administering the compositions described herein to the subject. In some embodiments, described herein is a method of increasing bone mineralization in a subject in need thereof, the method comprising administering the compositions described herein to the subject. In some embodiments, described herein is a method of increasing motor function in a subject in need thereof, the method comprising administering the compositions described herein to the subject. In some embodiments, described herein is a method of increasing quality of life in a subject in need thereof, the method comprising administering the compositions described herein to the subject. In some embodiments, the compositions comprise a viral vector for the delivery of an alkaline phosphatase.

### Disease and Disorders

HPP patients suffer from distinctive rickets and/or osteomalacia with a broad range of severity, as well as dental defects. There are seven major forms of HPP: life-threatening perinatal and infantile (OMIM #241500), benign perinatal, mild and severe childhood (OMIM #241510), adult (OMIM #136300), and odonto-HPP (OMIM #146300). Patients with perinatal HPP, the gravest form of HPP, often die in utero or soon after birth because of severe skeletal hypomineralization, respiratory failure due to thoracic cage dysplasia and hypoplastic lungs, and elevated intracranial pressure due to craniosynostosis. Dentoalveolar phenotypes, including premature exfoliation of primary teeth, periodontal disease, and enamel alternations, are commonly observed in patients with all forms of HPP.

In some embodiments, the subject has a soft bone disease. In some embodiments, the soft bone disease is hypophosphatasia (HPP). In some embodiments, the hypophosphatasia is pediatric hypophosphatasia or infantile hypophosphatasia. In some embodiments, the hypophosphatasia is late-onset hypophosphatasia.

In some embodiments, the disease comprises a dental disorder. All forms of HPP include dental involvement, with premature tooth loss being one of the most common manifestations. Characteristic dentoalveolar abnormalities observed in untreated Alpl^{-/-} mice include inhibition of tooth root acellular cementum formation, PDL detachment, and enamel and dentin mineralization defects. As described herein, qualitative and quantitative analyses suggest that teeth and associated periodontal tissues significantly improved from AAV8-TNAP-D₁₀-mediated gene therapy. AAV8-TNAP-D₁₀-treated Alpl^{-/-} mice showed normal formation and mineralization of molar and incisor enamel and dentin, and the restoration of acellular cementum, PDL cellularity, and PDL-cementum attachment. Treated Alpl^{-/-} mice also showed well-developed alveolar bone with mildly reduced mineral density. In some embodiments, the dental disorder comprises at least one of a dentoalveolar disorder, teeth hypomineralization, and a periodontal disorder.

There are more than 400 mutant alleles identified for the ALPL. The inheritance pattern of perinatal and infantile HPP is often autosomal recessive, with most patients being compound heterozygotes for pathogenic ALPL mutations that result in almost null ALP activity, but some are homozygous for recessive alleles and most adult and odonto-HPP patients harbor a single dominant-negative ALPL allele. In some embodiments, the subject has a mutation in the ALPL gene. In some embodiments, the subject has a PHOSPHO1 deficiency.

### Administration

In some embodiments, the viral vector is administered at least once. In some embodiments, administering the viral vector results in increased plasma alkaline phosphatases (ALP) activity. In some embodiments, ALP activity is increased for at least 1 week, 2 weeks, 3 weeks, 4 weeks, 2 months, 3 months, 4 months, 5 months, or 6 months. In some embodiments, ALP activity is increased for at least 1 week. In some embodiments, ALP activity is increased for at least 2 weeks. In some embodiments, ALP activity is increased for at least 3 weeks. In some embodiments, ALP activity is increased for at least 3 weeks. In some embodiments, ALP activity is increased for at least 4 weeks. In some embodiments, ALP activity is increased for at least 2 months. In some embodiments, ALP activity is increased for at least 3 months. In some embodiments, ALP activity is increased for at least 4 months. In some embodiments, ALP activity is increased for at least 5 months. In some embodiments, ALP activity is increased for at least 6 months.

In some embodiments, the viral vector is administered intramuscularly. In some embodiments, the viral vector does not diffuse away from the site of injection. In some embodiments, the viral vector does not diffuse away from the muscle. In some embodiments, after administration of the viral vector, the viral vector is not detected in the brain, gonads, or a combination thereof. In some embodiments, after administration of the viral vector, the viral vector is not detected in the brain. In some embodiments, after administration of the viral vector, the viral vector is not detected in the gonads.

In patients with HPP, with or without treatment, several types of ectopic calcifications are commonly observed: ocular calcification, nephrocalcinosis, and painful periarthritis. In some embodiments, following administering the viral vector, the subject does not develop an ectopic calcification. In some embodiments, the ectopic calcification is selected from the list of ocular calcification, nephrocalcinosis, painful periarthritis, or kidney stones. In some embodiments, the subject does not develop hypercalcemia. In some embodiments, the subject does not develop hypercalciuria. In some embodiments, the subject does not develop ectopic calcifications in a tissue of the subject. In some embodiments, the tissue is selected from the list consisting of kidneys, aorta, coronary arteries, and brain. In some embodiments, following administering the viral vector, the viral vector does not result in oncogenic effect in the subject.

### Compositions

In certain aspects, described herein are compositions for delivery of a therapeutic peptide to a subject in need thereof. In some embodiments, described herein is a viral vector for the delivery of an alkaline phosphatase to a subject in need thereof. In some embodiments, the viral vector comprises the sequence of a therapeutic peptide. In some embodiments, the therapeutic peptide comprises an alkaline phosphatase. In some embodiments, the therapeutic peptide comprises a mineral-targeted alkaline phosphatase. In some embodiments, the mineral-targeted alkaline phosphatase comprises tissue non-specific alkaline phosphatase (TNAP).

In some embodiments, the alkaline phosphatase comprises an alkaline phosphatase comprising the sequence of SEQ ID NO: 15. In some embodiments, the alkaline phosphatase comprises an alkaline phosphatase with at least 75%, 80%, 85%, 90%, 95%, 97.5%, 99% or 100% sequence identity with SEQ ID NO: 15.

In some embodiments, the mineral-targeted alkaline phosphatase comprises a sequence for bone targeting. The sequence for bone targeting may be linked to the C-terminus of the mineral-targeted alkaline phosphatase. The sequence for bone targeting may be linked to the N-terminus of the mineral-targeted alkaline phosphatase. The bone-targeting sequence may comprise at least one aspartate. The bone-targeting sequence may comprise at least 1, 2, 3, 4, 5, 6, 7, 8, 9, 10, 11, 12, 13, 14, 15 or more aspartates. In some embodiments, the bone-targeting sequence comprises a deca-aspartate (D₁₀) sequence. In some embodiments, the bone targeting sequence comprises the sequence DDDDDDDDDD (SEQ ID NO: 17). In some embodiments, the bone-targeting peptide comprises a repeat sequence of aspartate-serine-serine (DSS, SEQ ID NO: 18). In some embodiments, the bone-targeting sequence comprises at least 1, 2, 3, 4, 5, 6, 7, 8, or more repeats of aspartate-serine-serine. In some embodiments, the bone-targeting sequence comprises the sequence KRRTPVRE (SEQ ID NO: 19). In some embodiments, the bone-targeting sequence comprises the sequence KNFQSRSH (SEQ ID NO: 20). In some embodiments, the bone-targeting sequence comprises the sequence KTYASMQW (SEQ ID NO: 21). In some embodiments, the bone-targeting sequence comprises at least one sequence selected from the list consisting of SEQ ID NO: 17, SEQ ID NO: 18, SEQ ID NO: 19, SEQ ID NO: 20, and SEQ ID NO: 21.

The peptides described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes.

In some embodiments, the viral vector comprises a non-tissue specific promoter. In some embodiments, the tissue non-specific promotor comprises a CAG promotor. In some embodiments, the viral vector comprises a promoter with the sequence of SEQ ID NO 16. In some embodiments, the viral vector comprises a non-tissue specific promoter with at least with at least 75%, 80%, 85%, 90%, 95%, 97.5%, 99% or 100% sequence identity with SEQ ID NO. 16.

The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. In some embodiments, the vector comprises an adeno-associated virus (AAV) vector. In some embodiments, the AAV vector is an AAV1, AAV2, AAV3, AAV4, AAV5, AAV6, AAV7, AAV8, AAV9, or AAV10 vector. In some embodiments, the vector is an AAV8 vector.

Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

### Pharmaceutical Compositions

The compositions disclosed herein are formulated in any suitable manner for administration. Any suitable technique, carrier, and/or excipient is contemplated for use with the compositions disclosed herein. Non-limiting examples of cosmetic, dermatological, or pharmaceutically acceptable carriers and excipients suitable for formulation can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; Pharmaceutical Dosage Forms and Drug Delivery Systems, Eighth Ed. (Lippincott Williams & Wilkins 2004); and Muller, R. H., et al., Advanced Drug Delivery Reviews 59 (2007) 522-530, each of which is incorporated by reference in its entirety.

In some embodiments, the pharmaceutically acceptable carriers or excipients disclosed herein include, but are not limited to one or more: pH modifying agent (e.g., buffering agents), stabilizing agents, thickening agents, colorant agents, preservative agents, emulsifying agents, solubilizing agents, antioxidant agents, or any combination thereof. Other suitable compounds contemplated herein and within the knowledge of a practitioner skilled in the relevant art are found in the Handbook of Pharmaceutical Excipients, 4th Ed. (2003), the entire content of which is incorporated by reference herein.

In some embodiments, the compositions disclosed herein comprise one or more preservatives. The preservative, when utilized, is in an amount sufficient to extend the shelf-life or storage stability, or both, of the topical formulations disclosed herein. Exemplary preservatives include, but are not limited to: tetrasodium ethylene-diamine tetraacetic acid (EDTA), methyl, ethyl, butyl, and propyl parabens, benzophenone-4, methylchloroisothiazolinone, methylisothiazolinone, sodium benzoate, paraoxybenzoic acid esters, chlorobutanol, benzyl alcohol, phenylethylalcohol, dehydroacetic acid, sorbic acid, benzalkonium chloride (BKC), benzethonium chloride, phenol, phenylmercuric nitrate, and thimerosal.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. Furthermore, use of the term “including” as well as other forms, such as “include,” “includes,” and “included,” is not intended to be limited solely to the recited items. The section headings used herein are for organizational purposes only and are not to be construed as limiting the subject matter described.

The terms “effective amount” or “therapeutically effective amount,” as used herein, generally refer to a sufficient amount of an agent or a compound which will relieve, to some extent, or reduce the likelihood of the occurrence of one or more of the symptoms of the disease or condition being treated. The result can be reduction and/or alleviation of the signs, symptoms, or causes of a disease, or any other desired alteration of a biological system. The terms “effective amount” or “therapeutically effective amount” typically include, for example, a prophylactically effective amount. For example, a “prophylactically effective amount” is the amount of the composition described herein that is required to reduce the risk of absorption, transmission, or function of a pathogen in an individual or transmission of a pathogen to another individual.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject can be a mouse. The subject can be a TNAP knockout mouse. The subject may be diagnosed or suspected of being at high risk for a disease. In some cases, the subject is not necessarily diagnosed or suspected of being at high risk for the disease.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The terms “about” or “approximately,” as used herein, generally mean within an acceptable error range for the particular value as determined by one of ordinary skill in the art, which will depend in part upon how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 10% of a given value. Alternatively, particularly with respect to biological systems or processes, the term can mean within an order of magnitude, within 5-fold, and more preferably within 2-fold of a value.

### EXAMPLES

The following examples are illustrative and non-limiting to the scope of the compositions, methods, and formulations described herein.

### Example 1

### Mouse Model of Infantile HPP

TNAP knockout (Alpl^{-/-}) mice were created by inserting a Neo cassette into exon 6 of the mouse Alpl gene via homologous recombination. (Narisawa S, Frohlander N, Millan J L. Inactivation of two mouse alkaline phosphatase genes and establishment of a model of infantile hypophosphatasia. Dev Dyn. March 1997; 208(3):432-46.) Alpl^{-/-} mice phenocopy human infantile HPP, showing normal appearance and being indistinguishable from other siblings at birth. They displayed almost zero circulatory ALP activity, develop epileptic seizures, become cachectic, and die by 10 to 12 days postnatal (dpn) without additional supportive treatment. Alpl^{-/-} mice were maintained in a 12.5% C57Bl/6 and 87.5% 129J background and genotyped by PCR using genomic DNA extracted from toe samples within five days after birth. All animals (breeders, nursing mothers, pups, and weanlings) in this study were given free access to regular diet (2018 Teklad global 18% protein extruded rodent diets or 2019 Teklad global 19% protein rodent diets, Envigo, Indianapolis, IN, USA) with a standard level of vitamin B6, increased level of which was reported to improve the lifespan of Alpl^{-/-} mice. The institutional Animal Care and Use Committee (IACUC) approved all the animal studies.

Virus Vector Encoding the Human TNAP-D₁₀ cDNA

TNAP-D₁₀ contains recombinant human soluble TNAP (sALP), and a deca-aspartate (D₁₀) sequence at the C terminus, which enables TNAP to target mineralized tissues, such as bone and teeth. The human IgG1 Fc domain present in asfotase alfa to enable a one-step purification is absent from the product of this vector as purification is not required for viral-vector mediated in vivo expression. Recombinant AAV type 8 vector encoding TNAP-D₁₀ (AAV8-TNAP-D₁₀) under control of the CAG promoter was generated using the HEK293 cell line by the triple transfection method, purified, and then titrated as previously reported. Recombinant AAV type 8 vector encoding GFP (AAV8-GFP) was used as a control. Mice received a single injection of AAV8-TNAP-D₁₀ at a dose of 3×10¹¹ vector/genomes (vg)/body into the quadriceps femoris within 5 dpn. Eleven Alpl^{-/-} mice (male n=6, female n=5) and 14 wild type (WT) controls (male n=7, female n=7) were included in this study. After genotyping, all the Alpl^{-/-} mice received a single injection of AAV8-TNAP-D₁₀ at a dose of 3×10¹¹ vector genomes (vg)/body into the quadriceps femoris within 5 dpn. Three control WT mice received the same dose of AAV8-TNAP-D₁₀ to assess its effect on PPᵢ metabolism and soft tissue calcification when endogenous TNAP activity is present. Seven WT mice received the same amount of control AAV8-GFP vector and four WT mice were untreated; their data were combined and analyzed together as WT, due to lack of substantial differences between them. Mice were euthanized at 70 dpn by exsanguination, after intraperitoneal administration of Avertin. In mice, 70 dpn is the end of puberty, equivalent to a human age of 20 years, and root formation and cellular cementum formation have been completed. For the analysis of untreated Alpl^{-/-}, 17 WT, 7 heterozygote (Alpl^{+/-}), and 10 Alpl^{-/-} pups were collected at 10 dpn, euthanized by exsanguination after intraperitoneal administration of Avertin.

### Sample Collection and Biochemical Analyses

Body weight of the mice was measured at 35 and 70 dpn. Blood was collected from the orbital sinus of isoflurane-anesthetized mice using Pasteur pipets every 4 weeks after injection. Spot urine samples were collected simultaneously.

Blood was collected into two types of BD Microtainers coated with either clot activator or lithium heparin (Becton, Dickinson and Company, Franklin Lakes, NJ, USA). Blood for plasma collection and urine samples were placed on ice. Microtainers were then spun at 7,000 g for 10 minutes. Twenty μL of heparin-plasma was deproteinized using a Microcon-10 kDa Centrifugal Filter Unit with Ultracel-10 membrane (MilliporeSigma, Merck KGaA, Darmstadt, Germany), centrifuged at 14,000 g for 20 minutes. Urine was diluted 1:3 with 10 mM HEPES. Samples were stored at −80° C. for further analyses.

Plasma PPᵢ concentration was measured according to the protocol described previously. Five μL of deproteinized plasma sample and the PPᵢ standard ranging from 0.125 μM to 20 μM (Sodium pyrophosphate decahydrate, Sigma-Aldrich, St. Louis, MO, USA) was added to 45 μL of assay mixture containing 90 μM adenosine 5′ phosphosulfate sodium salt (APS) (Sigma-Aldrich), 22.5 μM MgCl₂, 11.25 mM HEPES with 0.9 U/mL recombinant Yeast ATP-sulfurylase/MET3 (R&D Systems, Inc., Minneapolis, MN, USA). The mixture was incubated at 37° C. for 30 minutes and heat-inactivated at 90° C. for 10 minutes. Ten μL of each sample were then transferred into a 96-well white-bottom plate and mixed with 50 μl of BacTiter-Glo Microbial Cell Viability Assay (Promega Corporation, Madison, WI, USA). Luminescence was measured by FilterMax F5 Multimode Microplate Readers (Molecular Devices, LLC., San Jose, CA, USA).

Serum ALP activity was measured using an enzymatic assay. Five μL of serum and recombinant human ALP standards (0.099 to 216.0 ng/mL) were mixed with 95 μL of 10 mM pNPP in diethanolamine (DEA) buffer (pH 9.8) containing 1.0 mM MgCl₂ and 20 μM ZnCl₂. The increase in A405 nm was measured using OptiMax Microplate Absorbance Reader (Molecular Devices, LLC., San Jose, CA, USA) for 15 minutes. QuantiChrom Calcium Assay Kit, QuantiChrom Creatinine Assay Kit, and QuantiChrom Urea Assay Kit (BioAssay Systems, Hayward, CA, USA) were used to measure serum and urine calcium, urine creatinine, and serum urea concentrations, respectively. Stanbio Phosphorus Liqui-UV (EKF Diagnostics-Stanbio Laboratory, Boerne, TX, USA) was used to measure serum and urine phosphorus concentrations.

Quantitative Polymerase Chain Reaction (qPCR)

Total RNA was extracted from the kidney using RNAeasy Plus Kit (Qiagen LLC, Germantown, MD, USA), and reverse transcription was carried out using PrimeScript RT Master Mix (Takara Bio USA, Inc., Mountain View, CA, USA). Real-time qPCR was performed in a 384-well plate in an Applied Biosystems 7900HT Fast Real-Time PCR system (Thermo Fisher Scientific, Waltham, MA, USA) using cDNA equivalent to 25 ng total RNA and DyNAmo Flash SYBR Green qPCR Kit (Thermo Fisher Scientific, Waltham, MA, USA). The reaction was run for 40 cycles at an initial temperature of 95° C. for 7 min and then at 95° C. for 10 s followed by 60° C. for 15 s. Ct values were determined by the software and the amplification of the target gene was normalized to that of 18S ribosomal RNA (Rn18s). Sequences of the primer pairs used for PCR are as follows: Alpl (NM_007431.3) F-CTGCCACTGCCTACTTGTGT (SEQ ID NO: 1) and R-GATGGATGTGACCTCATTGC (SEQ ID NO: 2); Ank (NM_020332.4) F-CTGCTGCTACAGAGGCAGTG (SEQ ID NO: 3) and R-GACAAAACAGAGCGTCAGCGA (SEQ ID NO: 4); Enpp1 (NM_001308327.1) F-TCACGCCACCGAGACTAAATA (SEQ ID NO: 5) and R-TGCAGTAGGGTGTCATGAAGG (SEQ ID NO: 6), Abcc6 (NM_018795.2) F-CATCTTGCCAGGAATCAACACT (SEQ ID NO: 7) and R-ACCAGGGACAAGCACAGGTA (SEQ ID NO: 8); Il6 (Interleukin 6) (NM_031168.2) F-CAAAGCCAGAGTCCTTCAGAGAG (SEQ ID NO: 9) and R-TTAGCCACTCCTTCTGTGACTCC (SEQ ID NO 10); Tnf (TNF-alpha) (NM_013693.3) F-CAGCCTCTTCTCATTCCTGCT (SEQ ID NO: 11) and R-GCCATTTGGGAACTTCTCATC (SEQ ID NO: 12); Rn18s (NR_003278.3) F-TTGATTAAGTCCCTGCCCTTTGT (SEQ ID NO: 13) and R-CGATCCGAGGGCCTCACTA (SEQ ID NO: 14).

### Radiography and Micro-Computed Tomography (μCT)

Radiographic images of entire skeletons and forelimbs, hindlimbs, and skulls were obtained with a Faxitron MX-20DC4 (Chicago, IL, USA), using energy of 20 kV. Lengths of the femur, tibia, humerus, and radius were measured using ImageJ (Rasband, W. S., ImageJ, National Institutes of Health, Bethesda, Maryland, USA, https://imagej.nih.gov/ij/, 1997-2018). Head measurements were performed using the following landmarks: (Liu J, Nam H K, Wang E, Hatch N E. Further analysis of the Crouzon mouse: effects of the FGFR2(C342Y) mutation are cranial bone-dependent. Calcif Tissue Int. May 2013; 92(5):451-66) nose length, the length from the rostal point of intersection of nasal bones to the caudal point of intersection of nasal bones; cranial length, the length from the rostal point of intersection of nasal bones to the median (midline) point of the posterior margin of the foramen magnum; cranial width, the length from the right joining of squamosal body to zygomatic process of squamous portion of temporal bone to the left counterpart.

After fixation in 4% paraformaldehyde/PBS solution, hemi-mandibles and femurs were scanned in a μCT 50 scanner (ScancoMedical, Bassersdorf, Switzerland) at 70 kV, 76 μA, 0.5 Al filter, 900 ms integration time, and 6 or 10 μm voxel dimension for mandibles and femurs, respectively. Reconstructed images were calibrated to 5 known densities of hydroxyapatite and analyzed using AnalyzePro (version 1.0; AnalyzeDirect, Overland Park, KS). For femurs, trabecular and cortical bones were segmented at 350 and 650 mgHA/cm³, respectively. The trabecular bone was traced using 50 slices (total of 0.5 mm) proximal to the distal femur growth plate to quantify bone volume (BV), total volume (TV), bone volume fraction (Tb. BV/TV), trabecular number (Tb. N), thickness (Tb. Th), spacing (Tb. Sp), connective density (1/mm³) and mineral density (Tb. BMD). For the cortical bone, 50 slices of the mid-femur of each bone was used to quantify cortical bone volume fraction (Ct. BV/TV), cortical thickness (Ct. Th), porosity, and mineral density (Ct. BMD).

The first mandibular molar and associated alveolar bone was quantitively analyzed as previously described. The alveolar bone region of interest (ROI) included the area between 240 μm mesial to the most mesial point of the first molar mesial root and 240 μm distal to the most distal point of the distal root. Enamel was segmented above 1,600 mg HA/cm³, while dentin/cementum and alveolar bone were segmented at 550-1,600 mg HA/cm³.

### Tissue Collection and Histological Studies

Skeletal and soft tissues were fixed in 4% paraformaldehyde/PBS solution and processed for histological analyses. Undecalcified fixed bone samples were placed in 30% sucrose/PBS solution and then cryo-embedded in Optimal Cutting Temperature (OCT) compound (Tissue-Tek, Torrance, CA, USA) in hexane dry ice bath and were sectioned by the Kawamoto method. (Kawamoto T. Use of a new adhesive film for the preparation of multi-purpose fresh-frozen sections from hard tissues, whole-animals, insects and plants. Arch Histol Cytol. May 2003; 66(2):123-43) The tibial and femur bones were placed in 0.125M EDTA/10% formalin (pH 7.3) solution for 7 days for decalcification and were then paraffin embedded. Soft organs were either paraffin embedded or embedded in an OCT in an ethanol dry ice bath. Hematoxylin and eosin (H&E), von Kossa, von Kossa/van Gieson, and Safranin O staining were performed according to standard methods. Tissue ALP activity was assayed by incubating the OCT-embedded sections in freshly mixed substrate solution made of one volume of 0.2 mg of Naphthol AS-MX phosphate disodium salt per ml of water and one volume of 1.2 mg FAST Violet B salt per mL of 0.2 M Tris-HCl (pH 8.9) at room temperature for 60 minutes, and counterstaining in methyl green solution. Slides were observed under IX81 Olympus Microscope (Olympus Corporation, Center Valley, PA, USA) or scanned by Aperio AT2 system (Leica Biosystems of Leica Microsystems Inc., Buffalo Grove, IL, USA). Bone volume fraction (BV/TV) was measured using ImageJ.

Left hemi-mandibles were fixed in Bouin's solution overnight, decalcified in an acetic acid/formalin/sodium chloride solution, processed for paraffin embedding and sectioned at 5 μm thickness in the coronal plane. Paraffin sections were stained with H&E to assess tooth and associated periodontium. Immunohistochemistry (IHC) procedures were performed as described previously. (Foster B L, Ao M, Salmon C R, Chavez M B, Kolli T N, Tran A B, et al. Osteopontin regulates dentin and alveolar bone development and mineralization. Bone. February 2018; 107:196-207.) Primary antibodies included: monoclonal rat anti-human alkaline phosphatase IgG (TNAP) (R&D systems, Minneapolis, MN, USA); polyclonal rabbit anti-mouse bone sialoprotein (BSP) IgG; and polyclonal LF-175 rabbit anti-mouse osteopontin (OPN) IgG. In situ hybridization was performed for mouse Alpl with (RNAscope 2.5 HD Detection reagent kit-RED assay, Advanced Cell Diagnostics) following the manufacturer's instructions as previously described. (Zhang H, Chavez M B, Kolli T N, Tan M H, Fong H, Chu E Y, et al. Dentoalveolar Defects in the Hyp Mouse Model of X-linked Hypophosphatemia. J Dent Res. April 2020; 99(4):419-28) For acellular cementum/PDL analysis, H&E-stained images captured with the same acquisition parameters were segmented using the color map function (5 Ramps) in ImageJ. This method is to pseudo-color the images to make differences between pixel values more apparent for improved tissue visualization. The values for acellular cementum and mantle dentin thickness represent the average of three linear measurements were taken at 90 μm, 100 μm, and 110 μm from the cemento-enamel junction (CEJ) using the ImageJ straight-line function. Mantle dentin is the outer layer, less mineralized dentin adjacent to acellular cementum. For assessing cellularity, a region of 5.5 mm² area was defined 100 μm apical to the CEJ for counting cells in the PDL space.

### Statistics

All the statistical analyses were performed using GraphPad Prism version 9.0.0 (GraphPad Software, San Diego, California, USA). Data are expressed as mean±standard deviation (SD) in charts. One-way analysis of variance (ANOVA) followed by Turkey's multiple comparisons test was performed to compare the differences among control WT, treated Alpl^{-/-}, and treated WT mice. Unpaired t-test with Welch's correction was performed to compare each gene expression in the kidneys of WT and treated Alpl^{-/-} mice. Significance was determined by P<0.05 and shown in the charts as *P<0.05, **P<0.01, ***P<0.001, and ****P<0.0001.

### Example 2

Improved Survival and Correction of Plasma PPᵢ in AAV8-TNAP-D₁₀-Treated Alpl^{-/-} Mice

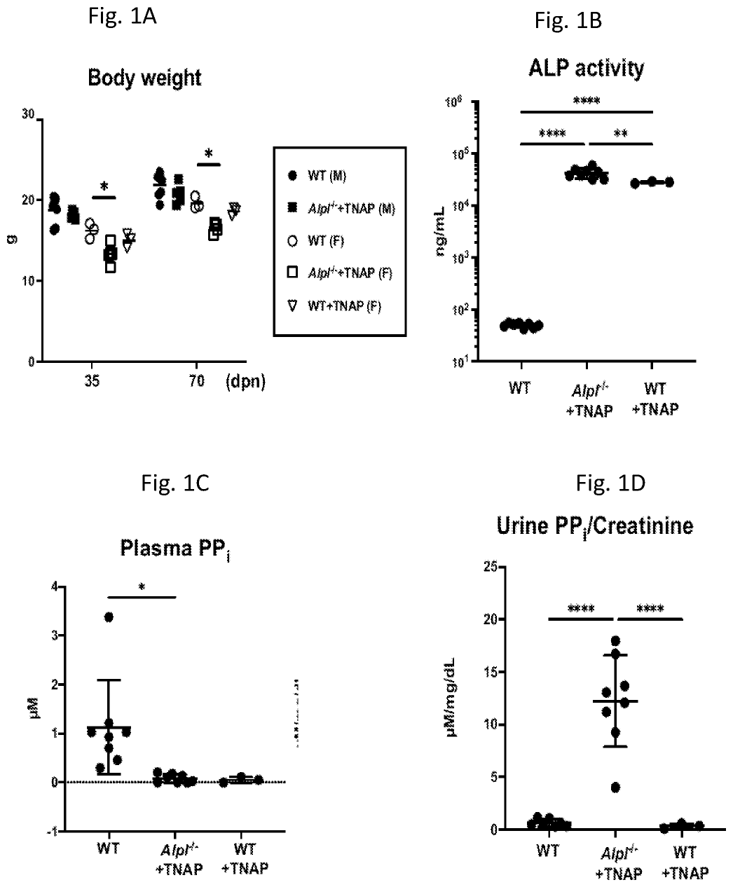

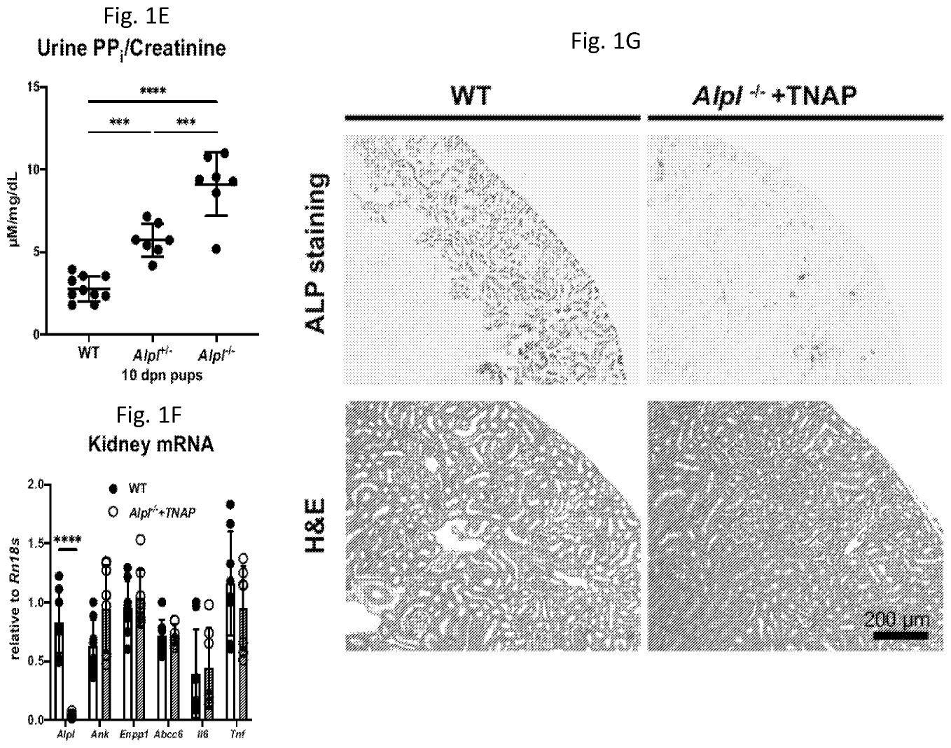

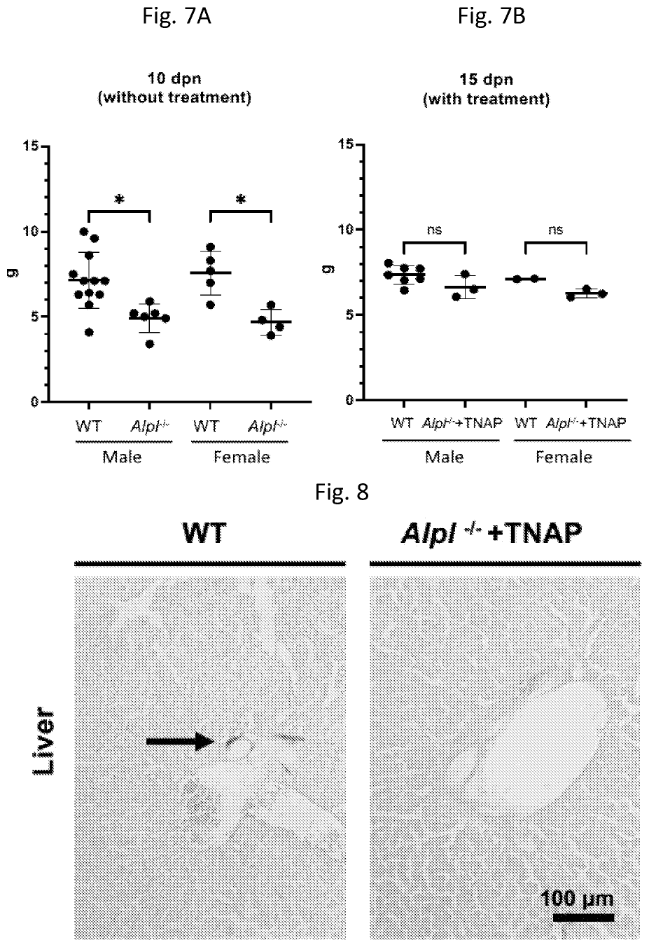

While untreated Alpl^{-/-} pups die within 10 to 12 days after birth, all Alpl^{-/-} mice treated with AAV8-TNAP-D₁₀ in this study did not develop epileptic seizures and were viable until the endpoint of the study at 70 dpn. The untreated Alpl^{-/-} pups did not feed well and were significantly smaller than WT littermates at 10 dpn (FIG. 7). In contrast, the Alpl^{-/-} mice injected with AAV8-TNAP-D₁₀ within 5 dpn showed catch-up growth, and their body weight was not significantly different from WT littermates at 15 dpn (FIG. 7). The body weight of AAV8-TNAP-D₁₀-treated male Alpl^{-/-} mice were comparable to their WT littermates, but treated female Alpl^{-/-} mice had lower body weight than their WT littermates both at 35 and 70 dpn (FIG. 1A).

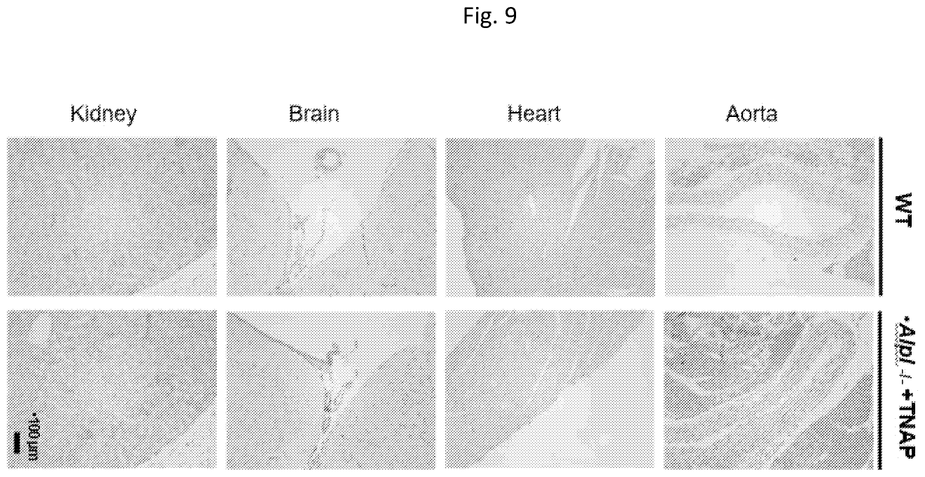

Serum ALP activity was significantly higher in Alpl^{-/-} and WT mice injected with AAV8-TNAP-D₁₀, approximately 500-800 times higher than that of control WT mice (FIG. 1B, Table 1). There were no significant differences in serum calcium, serum phosphorus, serum urea, urine calcium, or urine phosphorus concentrations among experimental groups (Table 1). While plasma PPᵢ concentrations were almost undetectable in the Alpl^{-/-} and WT mice treated with AAV8-TNAP-D₁₀ (FIG. 1C, Table 1), urine PPᵢ concentrations of AAV8-TNAP-D₁₀-treated Alpl^{-/-} mice remained significantly higher than WT mice at 70 dpn (FIG. 1D, Table 1). Elevated urine PPᵢ concentrations were also observed in untreated 10 dpn Alpl^{-/-} mice and Alpl^{+/-} mice (FIG. 1E). qPCR performed on RNA isolated from the kidney showed no expression of the Alpl gene in the Alpl^{-/-} mice, and no significant differences were observed in other genes related to PPᵢ metabolism (Ank, Enpp1, and Abcc6), nor in genes related to inflammation (Il6 and Tnf) (FIG. 1F, Table 2). Histochemical staining of the kidney and liver revealed no ALP activity in the renal proximal tubules and the branches of hepatic artery of AAV8-TNAP-D₁₀-treated Alpl^{-/-} mice (FIG. 1G and FIG. 8). Light microscopy did not reveal any apparent structural changes in the H&E-stained glomeruli or renal tubules (FIG. 1G). No ectopic calcifications were observed in the aorta, coronary arteries, brain, or kidney by 70 dpn (FIG. 9).

TABLE 1
MEASURED PARAMETERS AMONG EXPERIMENTAL GROUPS.

| | Genotype and treatment | | | |
|---|---|---|---|---|
| | | Alpl^{-/-} + | WT + | ANOVA |
| Parameters | WT | TNAP | TNAP | P-value |
| ALP, ng/mL | 50.02 ± 4.51 | 42287.44 ± 8599.55^{a} | 27994.17 ± 1074.31^{a} | <0.0001 |
| PPᵢ, μM | 1.128 ± 0.898 | 0.081 ± 0.080^{b} | 0.053 ± 0.045 | 0.0094 |
| Ca, mg/dL | 10.06 ± 0.80 | 10.57 ± 0.83 | 10.37 ± 0.81 | ns |
| P, mg/dL | 1.30 ± 1.10 | 2.35 ± 3.21 | 2.56 ± 2.00 | ns |
| BUN, mg/dL | 53.77 ± 6.76 | 52.24 ± 8.00 | 48.31 ± 7.24 | ns |
| Urine PPᵢ/Cre, | 0.641 ± 0.311 | 12.246 ± 4.080^{a} | 0.334 ± 0.198^{c} | <0.0001 |
| μM/mg/dL | | | | |
| Urine Ca/Cre | 0.676 ± 0.458 | 0.506 ± 0.196 | 0.585 ± 0.415 | ns |
| Urine P/Cre | 1.30 ± 1.10 | 2.35 ± 3.21 | 2.56 ± 2.00 | ns |

ns: no statistical significance between genotypes
^{a}P < 0.0001 compared to WT,
^{b}P < 0.05 compared to WT,
^{c}P < 0.0001 compared to Alpl^{-/-} + TNAP

TABLE 2
SUMMARY OF QPCR ANALYSIS OF THE KIDNEY

| | Genotype and treatment | | Welch's t-test |
|---|---|---|---|
| Genes | WT | Alpl^{-/-} + TNAP | P-value |
| Alpl | 0.83 ± 0.25 | 0.04 ± 0.02 | <0.0001 |
| Ank | 0.62 ± 0.21 | 0.94 ± 0.35 | 0.0608 |
| Enpp1 | 0.95 ± 0.21 | 1.04 ± 0.23 | 0.4732 |
| Abcc6 | 0.71 ± 0.13 | 0.72 ± 0.08 | 0.8705 |
| Il6 | 0.39 ± 0.35 | 0.44 ± 0.32 | 0.8155 |
| Tnf | 1.16 ± 0.41 | 0.95 ± 0.33 | 0.3244 |

Delta-delta Ct method was used to calculate relative gene expressions with Rn18s as a reference gene. Data are presented as mean ± standard deviation. Statistical analysis was performed by unpaired t-test with Welch's correction.

Improved Bone Microstructure in AAV8-TNAP-D₁₀-Treated Alpl^{-/-} Mice

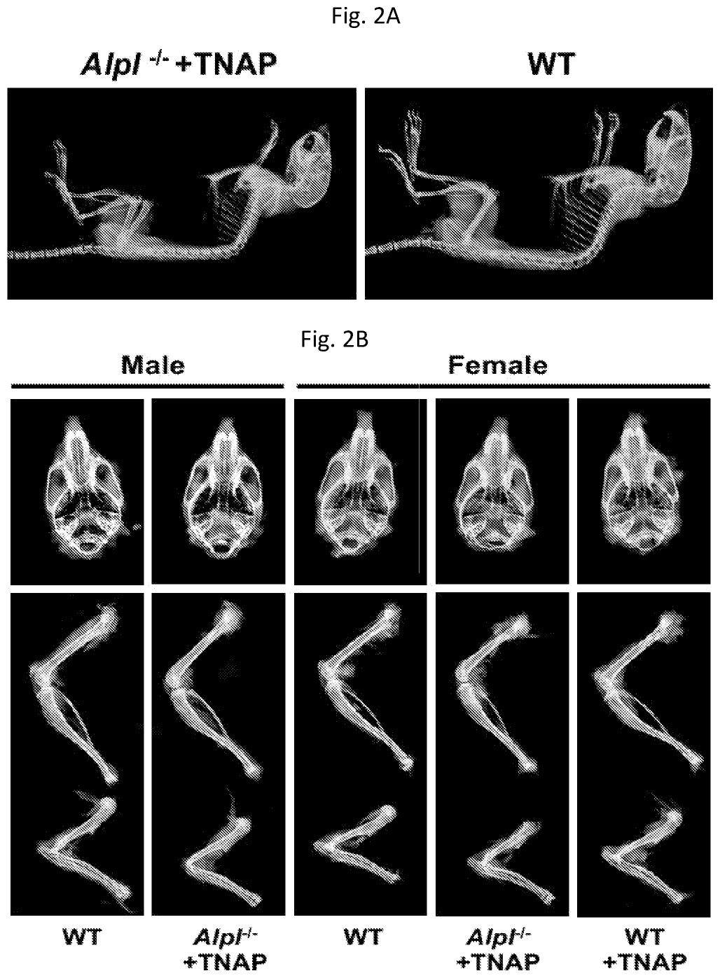

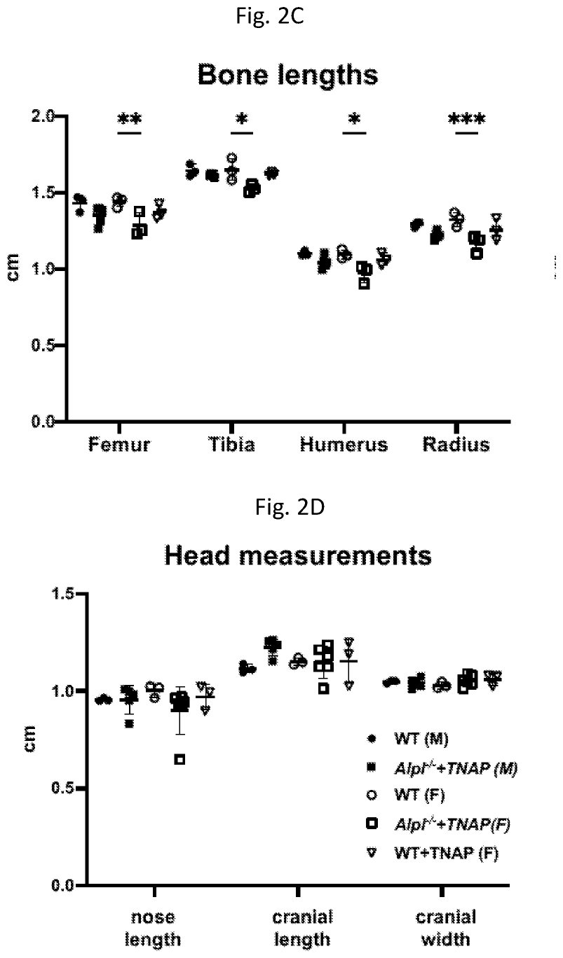

In previous studies, untreated 20-23 dpn Alpl^{-/-} mice demonstrated profound skeletal abnormalities including reduced tissue mineral density and bone fractures. Radiography of 70 dpn Alpl^{-/-} mice treated with AAV8-TNAP-D₁₀ revealed grossly normal skeletal development comparable to WT littermates (FIG. 2A). Treated Alpl^{-/-} mice did not show features characteristic of rickets or osteomalacia, such as bowing of the long bones, enlargement of the ends of epiphyses, or fractures (FIG. 2B). Long bone lengths of treated male Alpl^{-/-} mice were not significantly different from those of WT mice, while treated female Alpl^{-/-} mice were significantly smaller than their WT littermates (FIG. 2C). Treated Alpl^{-/-} mice did not show craniosynostosis (FIG. 2B). Nose length, cranial length, and cranial width were not significantly different among each treatment group, while the nose and cranial lengths varied among individuals, especially in the AAV8-TNAP-D₁₀-treated Alpl^{-/-} mice (FIG. 2D, Table 3).

TABLE 3
SUMMARY OF HEAD MEASUREMENTS OF 70 DPN MICE

| | Genotype and treatment | | | |
|---|---|---|---|---|
| Parameters | | Alpl^{-/-} + | WT + | Welch's t-test |
| (Male) | WT | TNAP | TNAP | P-value |
| Nose length | 0.96 ± 0.01 | 0.95 ± 0.06 | N/A | 0.9717 |
| Cranial length | 1.11 ± 0.02 | 1.23 ± 0.04 | N/A | 0.0033 |
| Cranial width | 1.05 ± 0.01 | 1.04 ± 0.02 | N/A | 0.7144 |

| | Genotype and treatment | | | |
|---|---|---|---|---|
| Parameters | | Alpl^{-/-} + | WT + | ANOVA |
| (Female) | WT | TNAP | TNAP | P-value |
| Nose length | 1.00 ± 0.03 | 0.90 ± 0.11 | 0.97 ± 0.05 | 0.3211 |
| Cranial length | 1.15 ± 0.01 | 1.15 ± 0.07 | 1.15 ± 0.09 | 0.9952 |
| Cranial width | 1.03 ± 0.01 | 1.05 ± 0.03 | 1.06 ± 0.03 | 0.3647 |

Data are presented as mean ± standard deviation. Statistical analysis was performed by unpaired t-test with Welch's correction in male and one-way ANOVA in female.
N/A: NO APPLICABLE DATA

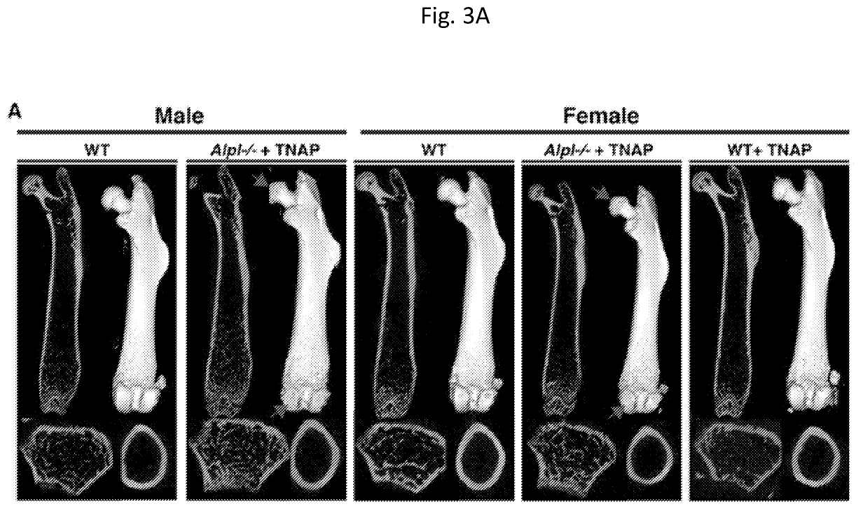

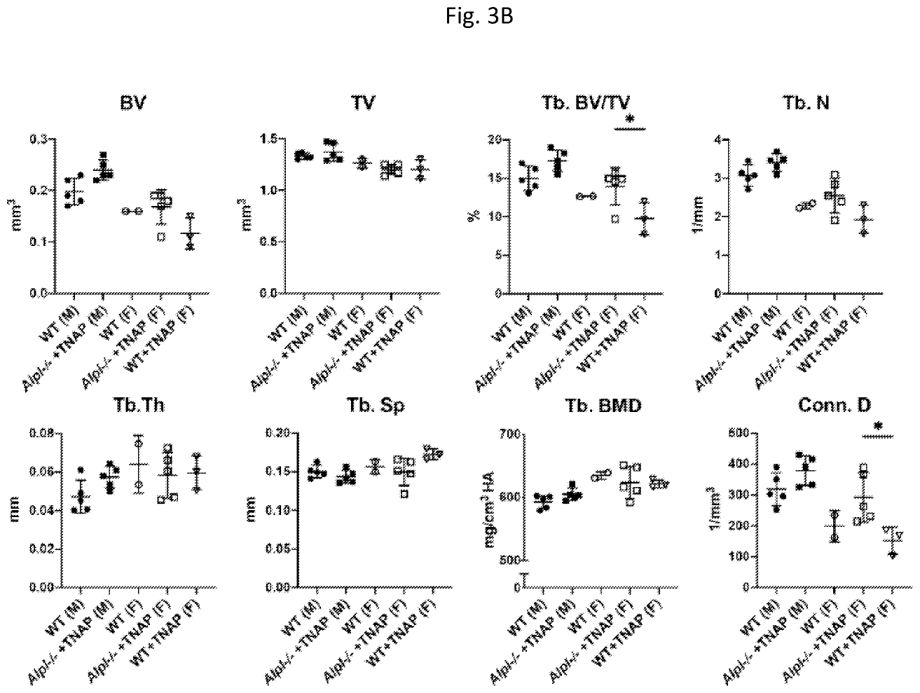

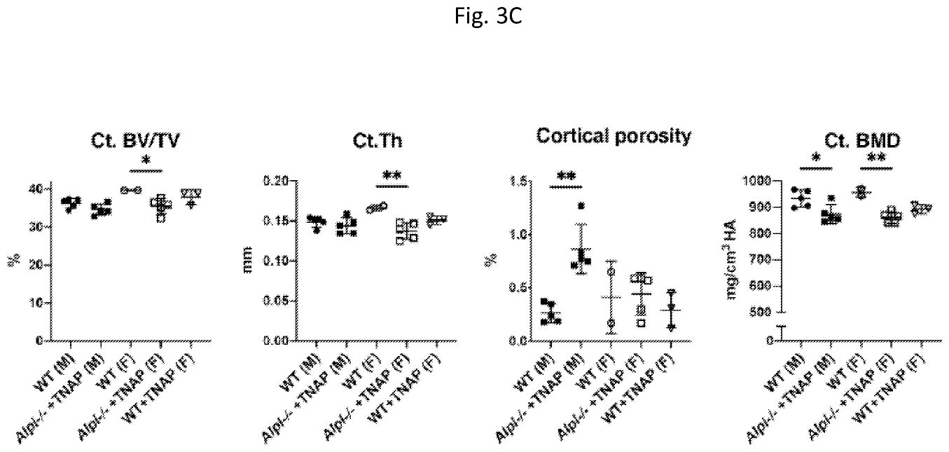

Three-dimensional μCT renderings of AAV8-TNAP-D₁₀-treated Alpl^{-/-} femurs showed bone morphology similar to WT femurs; however, the bone was ˜7-9% shorter in treated Alpl^{-/-} females compared with WT controls (FIG. 2C, 3A). Notably, treated Alpl^{-/-} mice had abnormal articular surfaces the femur joints compared with that of WT controls. Quantitative μCT analysis showed similar bone trabecular and cortical parameters in treated male and female Alpl^{-/-} femurs compared with WT controls. Metaphyses of distal femurs showed no significant differences in total volume (TV), bone volume (BV), or trabecular bone volume fraction (Tb. BV/TV) amongst groups (FIG. 3B). However, treated Alpl^{-/-} females showed increased Tb. BV/TV associated with improved trabecular connectivity compared with treated WT mice (FIG. 3B). No significant differences were detected in trabecular number (Tb. N), thickness (Tb. Th), spacing (Tb. Sp), or bone mineral density (Tb. BMD) among the groups. However, AAV8-TNAP-D₁₀₋treated Alpl^{-/-} females showed reduced cortical BV/TV associated with decreased cortical thickness. Treated Alpl^{-/-} males showed increased cortical porosity but this did not significantly affect Ct. BV/TV. Also, AAV8-TNAP-D₁₀ failed to completely restore the cortical bone mineral density (Ct. BMD), both in males and females (FIG. 3C), suggesting an incomplete rescue of cortical bone defects.

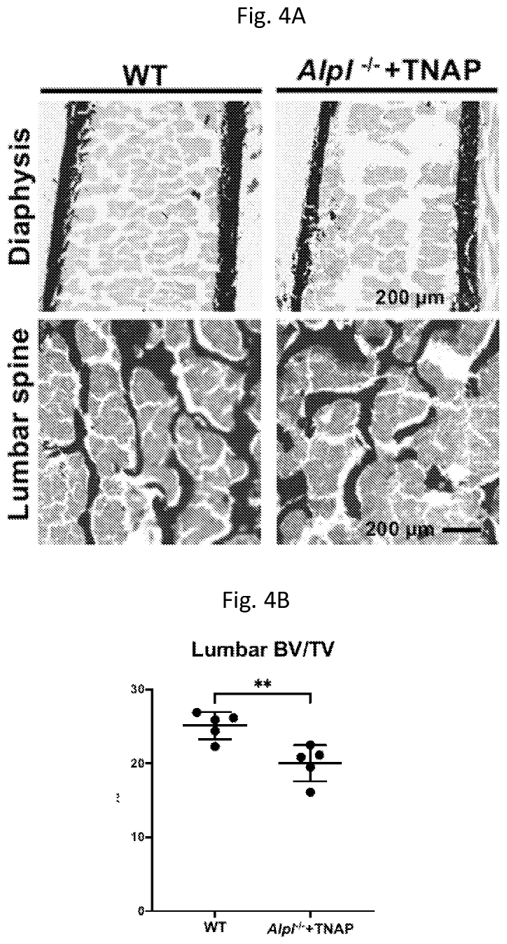

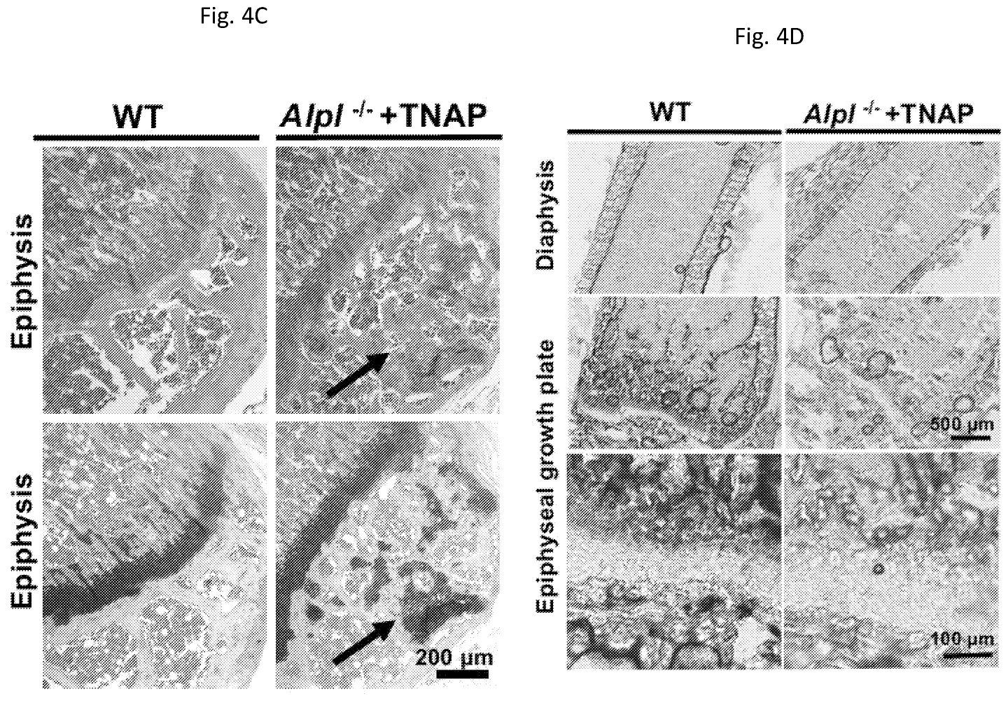

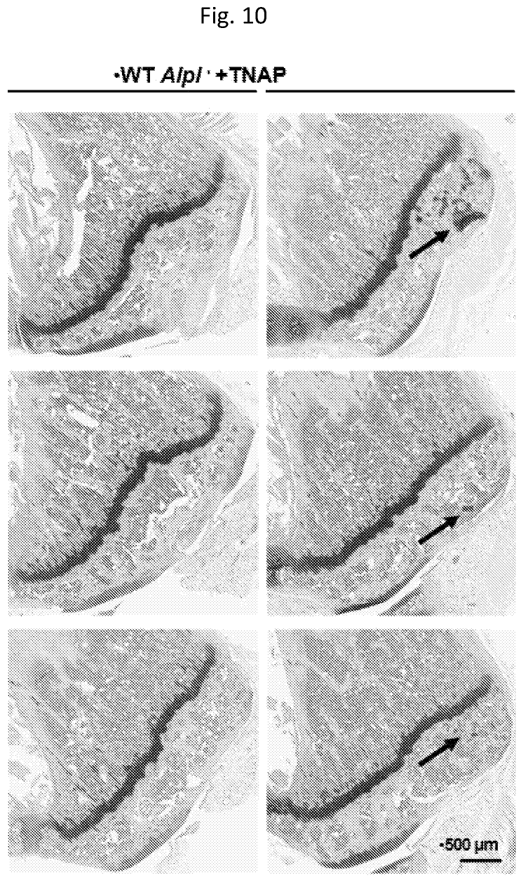

Von Kossa/van Gieson staining of femurs and lumbar spines showed no measurable osteoid surface (FIG. 4A), but the BV/TV values of lumbar spines of AAV8-TNAP-D₁₀-treated Alpl^{-/-} mice were significantly lower than that of WT mice (FIG. 4B, WT n=5; male n=2, female n=3, KO n=3; male n=1, female n=2). H&E and Safranin O staining of the decalcified tibias did not show the expansion of the tibial epiphyseal growth plate but showed an abnormal distribution of chondrocytes in secondary ossification centers in AAV8-TNAP-D₁₀-treated Alpl^{-/-} mice (FIG. 4C, FIG. 10). Histochemical staining of the femurs revealed strong ALP activity in the hypertrophic zone of the epiphyseal growth plate, metaphysis, and diaphysis of WT mice, while only subtle ALP activity was observed in the growth plate and diaphysis of AAV8-TNAP-D₁₀-treated Alpl^{-/-} mice (FIG. 4D).

AAV8-TNAP-D₁₀ Prevents HPP-Associated Dentoalveolar Defects in Alpl^{-/-} Mice

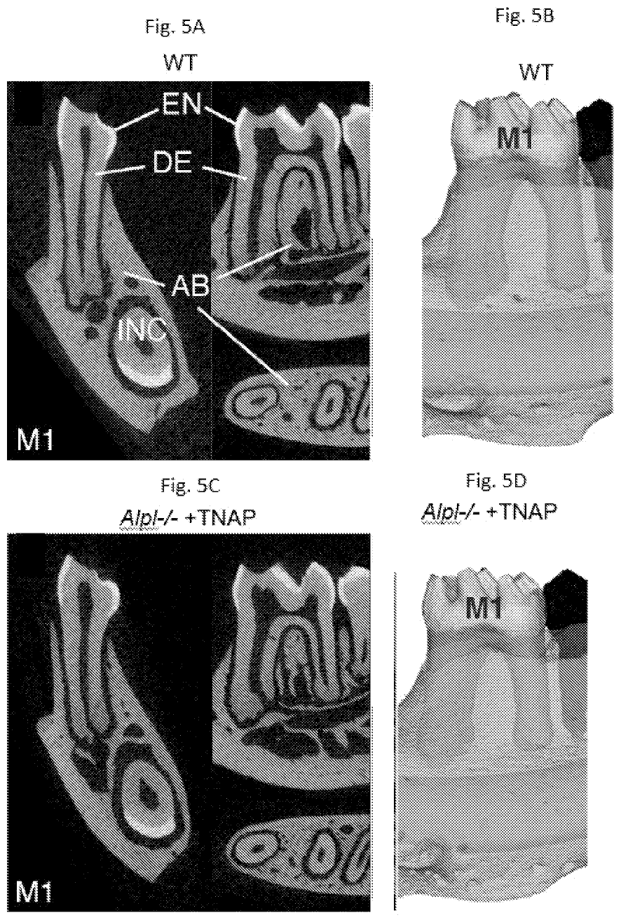

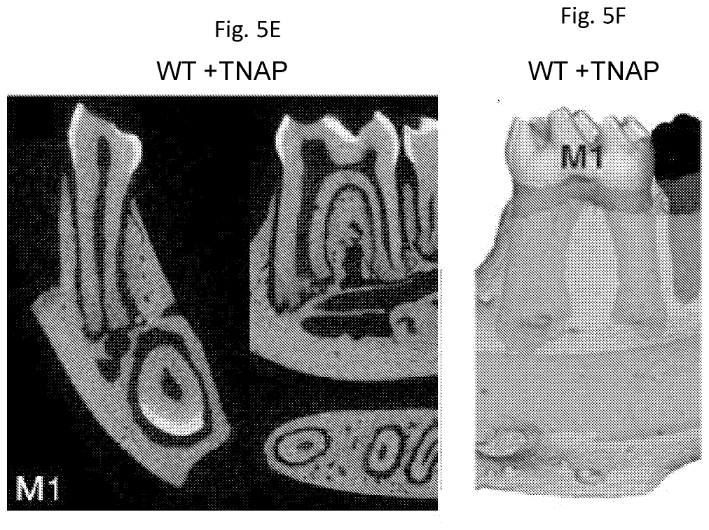

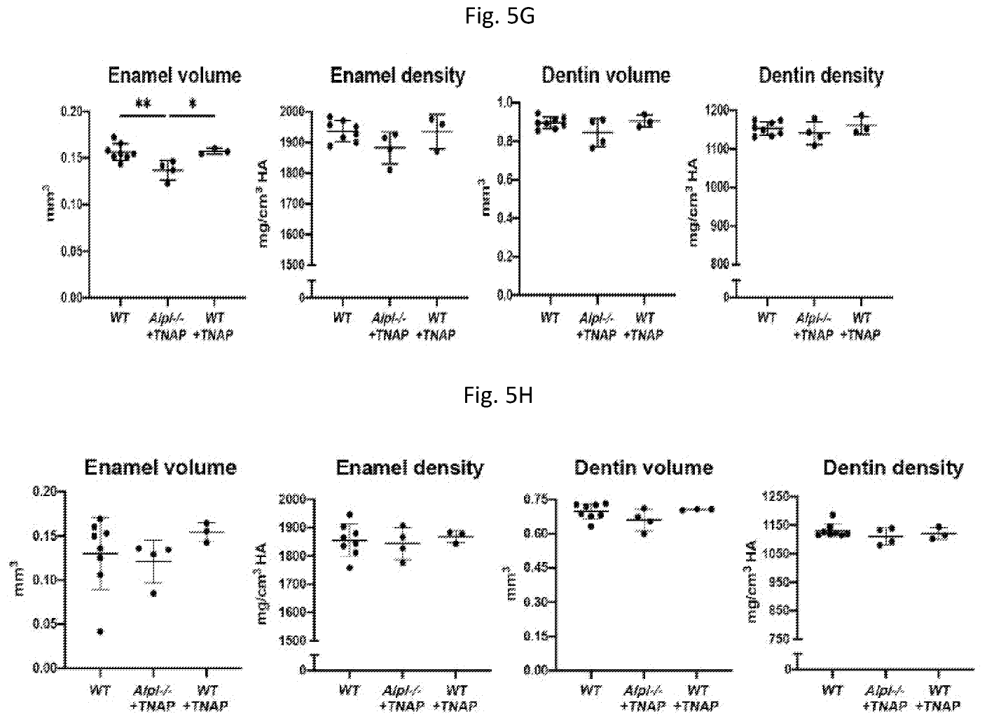

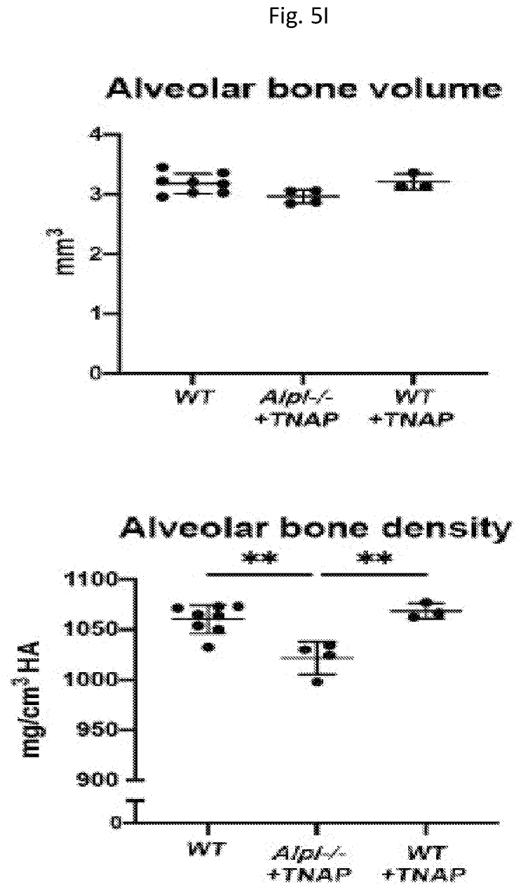

Data from male and female dentoalveolar tissues were combined together due to lack of substantial sex-related differences in previous reports of HPP mouse models, and because sex-related trends in these data were not found. Compared to untreated and treated WT controls, first mandibular molars and surrounding alveolar bone of AAV8-TNAP-D₁₀-treated Alpl^{-/-} mice appeared grossly normal (FIG. 5A-5F). Enamel volumes were 12-14% reduced in molars of AAV8-TNAP-D₁₀-treated Alpl^{-/-} compared to WT groups, though no significant differences were observed in enamel mineral density (FIG. 5G). No differences in molar dentin volume or density were observed between AAV8-TNAP-D₁₀-treated Alpl^{-/-} and WT groups (FIG. 5G). Continually erupting incisors of Alpl^{-/-} mice, which harbor mineralization defects previously shown to be relatively resistant to treatment, showed no differences in the volumes or densities of enamel or dentin compared to WT groups (FIG. 5H). Alveolar bone showed no differences in the volume between AAV8-TNAP-D₁₀-treated Alpl^{-/-} mice and WT controls, though exhibited ˜4% lower mineral density than WT groups (FIG. 5I).

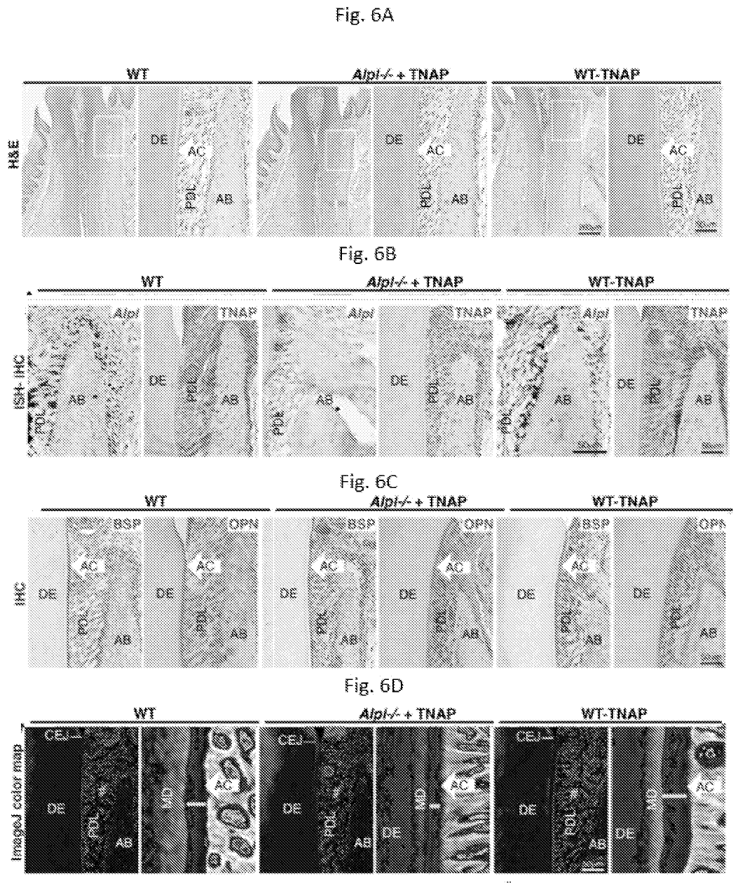

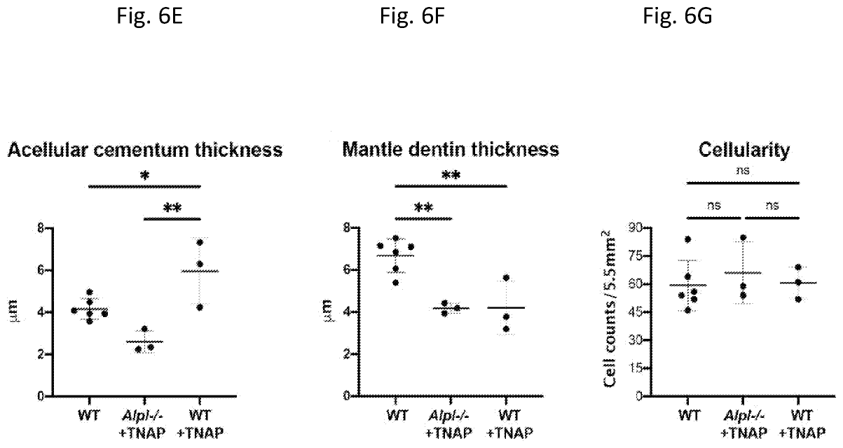

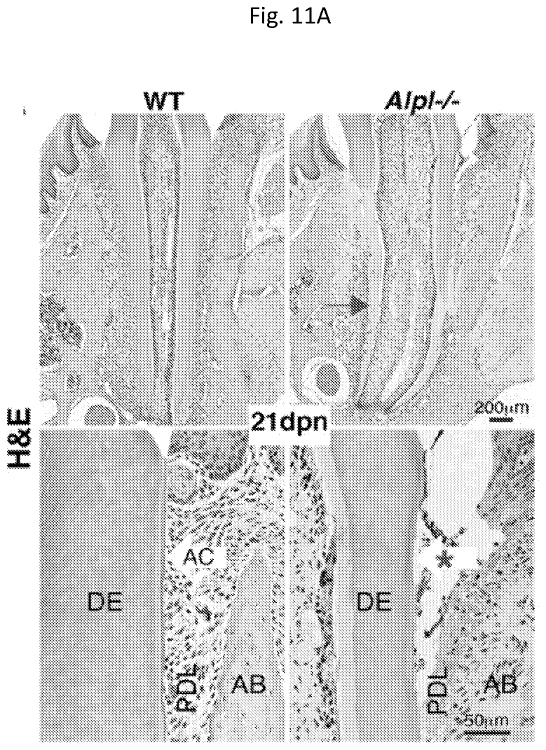

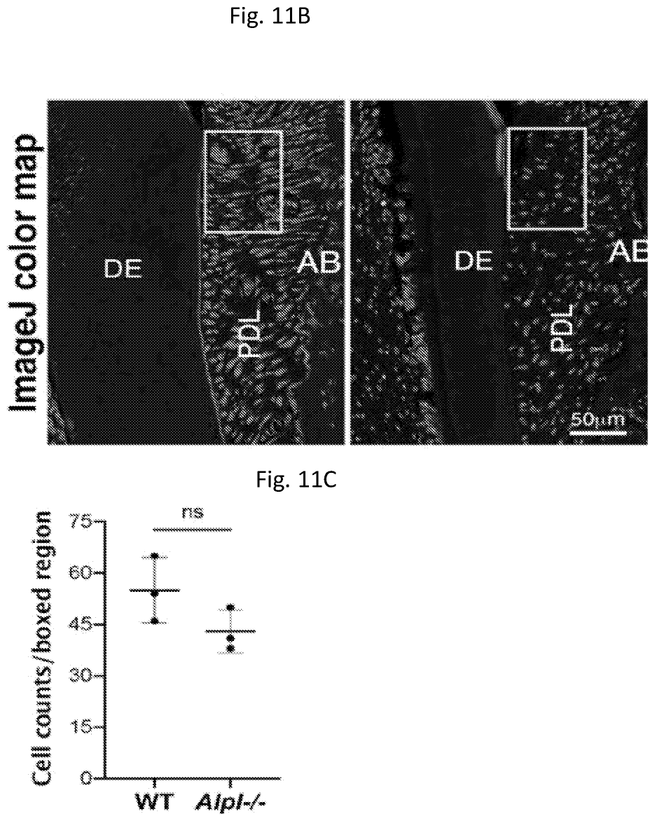

Histology of AAV8-TNAP-D₁₀-treated Alpl^{-/-} mouse mandibles revealed normal tooth structures largely indiscernible from those of WT controls, with similar morphology, tissue organization, presence of acellular cementum on root surfaces, and periodontal attachment (FIG. 6A). While ISH showed no endogenous Alpl expression in AAV8-TNAP-D₁₀-treated Alpl^{-/-} mouse tissues, IHC detected low level TNAP localized in the PDL (FIG. 6B), suggesting the contribution of AAV8-mediated TNAP for amelioration of defects. AAV8-TNAP-D₁₀-treated Alpl^{-/-} mice showed BSP and OPN immunostaining comparable to WT controls, confirming the identity of acellular cementum (FIG. 6C). Histomorphometry revealed no significant differences in acellular cementum thickness of treated Alpl^{-/-} vs. WT mouse molars, though TNAP-D₁₀-treated WT teeth showed thickened acellular cementum (FIG. 6D, 6E). Outermost mantle dentin, which is enlarged and hypomineralized in humans with HPP and whose mineralization is delayed in Alpl^{-/-} mice, was reduced in both treated Alpl^{-/-} and WT mice (FIG. 6D, 6F). While untreated Alpl^{-/-} mice showed PDL hypocellularity associated with defects in acellular cementum and PDL attachment (FIG. 11), normal cellularity returned with treatment (FIG. 6D, 6G) and periodontal structures appeared intact.

### Example 3: Analysis of Mice Under AAV8-TNAP-D10 Treatment

### Methods

Virus Vector Encoding the Human TNAP-D10 cDNA

Recombinant adeno-associated virus vector serotype 8 (AAV8) encoding mineral-targeted TNAP (AAV8-TNAP-D₁₀) was generated using the HEK293 cell line. Recombinant AAV8 vector encoding GFP (AAV8-GFP) was used as a control. Eight weeks old Alpl^{Prx1/Prx1} and WT siblings mice, as well as Phospho1^{-/-} and WT littermates, received a single intramuscular injection of AAV8-TNAP-D₁₀ or AAV8-GFP vector as control at a dose of 3×10¹¹ vector genomes (vg)/body into the quadriceps femoris.

### Induction of Uremia

Eight weeks old Alpl^{Prx1/Prx1} and WT siblings mice were fed with a diet containing adenine (0.2%) for four weeks, and adenine+high phosphorus diet (0.2% adenine and 1.8% phosphorus) for another 4 weeks, protocol adapted from Tani et al., Inhibition of tissue-nonspecific alkaline phosphatase protects against medial arterial calcification and improves survival probability in the CKD-MBD mouse model. J Pathol. January 2020; 250(1):30-41, to induce a state of uremia that recapitulates CKD, which leads to vascular calcification. The animals were monitored weekly by measuring body weight and observing clinical signs such as posture and gait, difficulty of eating, etc. Animals received a single dose of AAV8-TNAP-D₁₀ or AAV8-GFP treatment on the same day as the induction of uremia. After 8 weeks, mice were sacrificed, and soft organs such as kidneys, aorta, and heart, were harvested from each animal to assess the status of calcification and whether the treatment can worsen the ectopic calcification.

### Biochemical Analysis

Body weight of the mice was first measured, and then they were anesthetized with isoflurane for blood (plasma/serum) collection from the orbital sinus of mice using Pasteur pipets before the treatment and 60 days after injection for late-onset HPP mice, and after 45 and 90 days of treatment for the pseudo-HPP mice. Blood samples were centrifuged at 7,500 rpm for 10 minutes. For PPᵢ analysis, twenty μL of heparin-plasma was deproteinized using a Microcon-10 kDa Centrifugal Filter Unit with Ultracel-10 membrane (MilliporeSigma, Merck KGaA, Darmstadt, Germany), centrifuged at 14,000 g for 20 minutes. Samples were stored at −80° C. for further analyses.

Plasma PPᵢ concentrations were measured. Five μL of deproteinized plasma sample and the PPᵢ standard ranging from 0.125 μM to 20 μM (Sodium pyrophosphate decahydrate, Sigma-Aldrich, St. Louis, MO, USA) was added to 45 μL of assay mixture containing 90 μM adenosine 5′ phosphosulfate sodium salt (APS) (Sigma-Aldrich), 22.5 μM MgCl₂, 11.25 mM HEPES with 0.9 U/mL recombinant Yeast ATP-sulfurylase/MET3 (R&D Systems, Inc., Minneapolis, MN, USA). The mixture was incubated at 37° C. for 30 minutes and heat-inactivated at 90° C. for 10 minutes. Ten μL of each sample were then transferred into a 96-well white-bottom plate and mixed with 50 μL of BacTiter-Glo Microbial Cell Viability Assay (Promega Corporation, Madison, WI, USA). Luminescence was measured by FilterMax F5 Multimode Microplate Readers (VWR International, LLC., Radnor, PA, USA). Serum ALP activity was measured using an enzymatic assay. Five μL of serum and recombinant human ALP standards (0.099 to 216.0 ng/mL) were mixed with 95 μL of 10 mM pNPP in diethanolamine (DEA) buffer (pH 9.8) containing 1.0 mM MgCl₂ and 20 μM ZnCl₂. The absorbance of the kinetics assay was measured at 405 nm using OptiMax Microplate Absorbance Reader (Molecular Devices, LLC., San Jose, CA, USA) for 15 minutes. QuantiChrom Calcium Assay Kit and QuantiChrom Urea Assay Kit (BioAssay Systems, Hayward, CA, USA) were used to measure serum calcium, and serum urea concentrations, respectively. Stanbio Phosphorus Liqui-UV (EKF Diagnostics-Stanbio Laboratory, Boerne, TX, USA) was used to measure serum phosphorus concentrations.

### Radiography and Micro-Computed Tomography (Micro-CT)

Radiographic images of the whole skeleton as well as the isolated bones such as the skull, vertebra, long bones, and hemi-mandibles, were obtained with the Faxitron MX-20DC4 radiograph system (Chicago, IL, USA), using the energy of 20 kV.

Before micro-CT analysis, long bones and right hemimandibles were fixed in 4% paraformaldehyde/PBS solution. Femurs were scanned using a Sky Scan 1172 scanner (Bruker Micro-CT, Kontich, Belgium) at 55 kV, 181 μA, 0.5 mm Al filter, 280 ms integration time, and m voxel dimension. Micro-CT images were reconstructed in NRecon software, and reconstructed images were calibrated to three known densities of hydroxyapatite and analyzed using AnalyzePro (version 1.0; AnalyzeDirect, Overland Park, KS). Trabecular and cortical bones of femurs were segmented at 400 and 550 mgHA/cm³, respectively. Micro-CT analysis of femurs was performed. Briefly, the trabecular bone was traced using 50 slices (total of 0.5 mm) proximal to the distal femur growth plate to quantify bone volume (BV), total volume (TV), bone volume fraction (Tb. BV/TV), trabecular number (Tb. N), thickness (Tb. Th), spacing (Tb. Sp), connective density (1/mm³), and mineral density (Tb. BMD). For the cortical bone, 50 slices of the mid-femur of each bone were used to quantify cortical bone volume fraction (Ct. BV/TV), cortical thickness (Ct. Th), marrow area, and mineral density (Ct. BMD).

Hemimandibles were scanned using a micro-CT 50 scanner (Scanco Medical, Bassersdorf, Switzerland) at 70 kV, 76 μA, 0.5 Al filter, 900 ms integration time, and 6 μm voxel dimension. Reconstructed images were calibrated to five known densities of hydroxyapatite and analyzed using AnalyzePro (version 1.0; AnalyzeDirect, Overland Park, KS). Micro-CT analysis of teeth was performed using the first mandibular molar and associated alveolar bone. The alveolar bone region of interest was defined to include 240 μm mesial to the most mesial point of the first molar mesial root and 240 μm distal to the most distal point of the distal root. Alveolar bone and dentin/cementum were segmented at 650-1,600 mg/cm³ HA, while enamel was segmented above 1,600 mg/cm³ HA.

### Tissue Collection and Processing for Histological Studies

Treated AlplPrx1/Prx1 and Phospho1−/− mice were euthanized 60 days and 90 days, respectively, by exsanguination after an intraperitoneal administration of Avertin, and skeletal/dental tissues were collected, fixed in 4% paraformaldehyde/PBS or Boiun's solution, and processed for histological analysis. The long bones and vertebrae were placed in 0.125M EDTA/10% formalin (pH 7.3) solution for 7 days for decalcification and then were paraffin-embedded and sectioned at 5 μm thickness. Hemi-mandibles were fixed in Bouin's solution, decalcified in an acetic acid/formalin/sodium chloride solution, processed for paraffin embedding, and sectioned at 5 μm thickness. Hematoxylin and eosin (H&E) and alizarin red staining were performed according to standard methods. Slides were scanned by the Aperio AT2 system (Leica Biosystems of Leica Microsystems Inc., Buffalo Grove, IL, USA).

### Statistical Analysis

All statistical analyses were performed using GraphPad Prism version 9.0.0 (GraphPad Software, San Diego, California, USA) using one-way ANOVA followed by Tukey's multiple comparisons test for comparisons with more than two groups or Unpaired t-test for comparisons between two groups. Values were expressed as the mean±SD. Differences were statistically significant at *p<0.05.

### Results

### Biochemical Analysis of Mice Under AAV8-TNAP-D10 Treatment

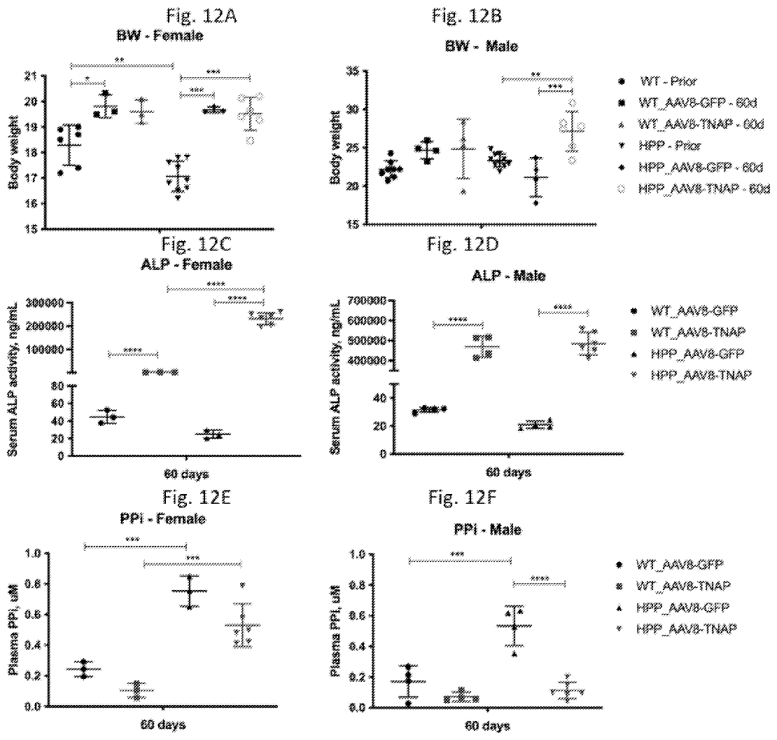

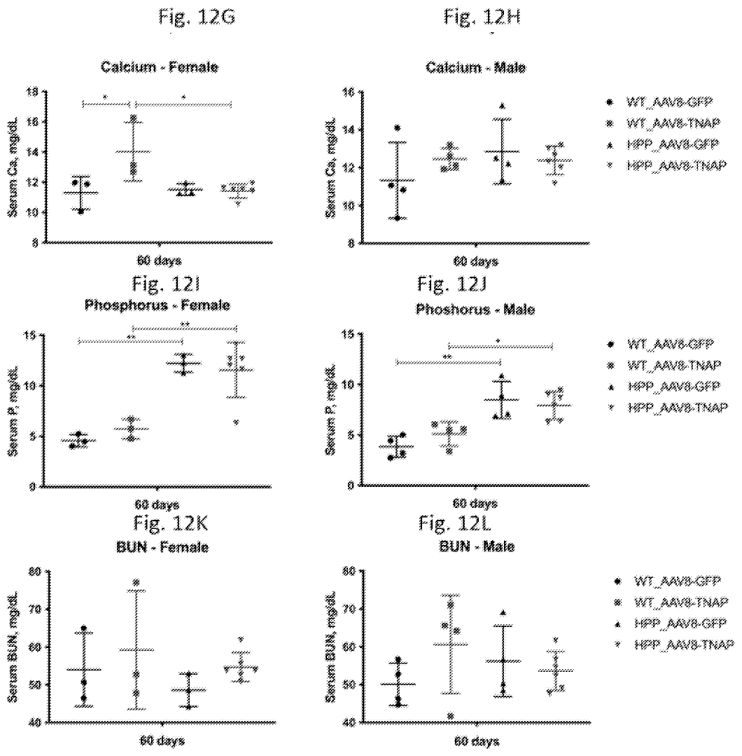

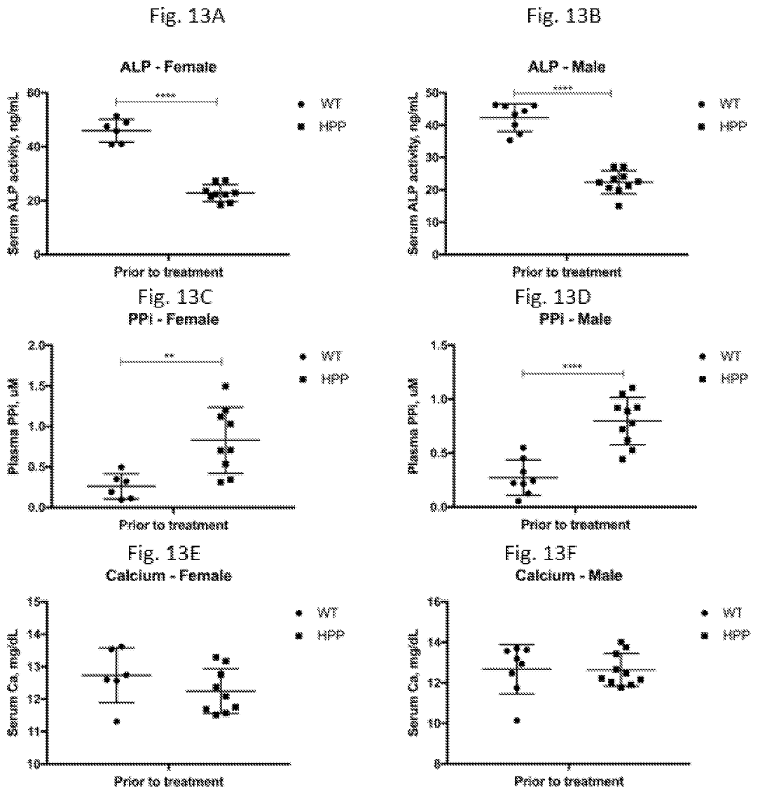

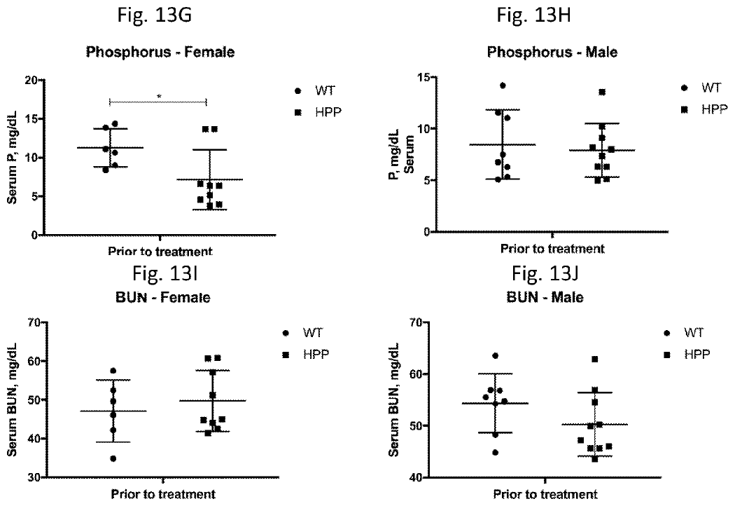

Body weight and biochemical markers in serum and/or plasma were monitored from adult HPP and WT mice prior to treatment and 60 days after the AAV8-TNAP-D₁₀ injection. Body weights of female adult HPP mice were slightly lower than WT littermates prior to the injection. After 60 days of treatment, there was an increase in body weight while in males no differences were observed. Male adult HPP mice showed body weight increase after AAV8-TNAP-D₁₀ treatment when compared to HPP AAV8-GFP injected mice (FIG. 12 A-12B). Serum ALP activity results were significantly lower and PPi was significantly higher in female and male adult HPP mice than WT mice prior to treatment (FIG. 13A-13D, Table 4). There were no differences in serum calcium, blood urea nitrogen (BUN), and phosphorus levels in HPP and WT mice before treatment, except for female HPP, in which lower phosphorus levels were observed when compared to WT mice before injection (FIG. 13E-13J, Table 4).

TABLE 4
Biochemical analysis of late-onset HPP
and WT treated mice before treatment.

| Biochemical | Prior to treatment | | ANOVA |
|---|---|---|---|
| markers | WT | HPP | P-value |
| ALP, ng/mL | F: 45.95 ± 4.24 | F: 22.77 ± 3.14 | <0.0001**** |
| | M: 42.36 ± 4.28 | M: 22.35 ± 3.58 | <0.0001**** |
| PPᵢ, μM | F: 0.262 ± 0.15 | F: 0.828 ± 0.40 | 0.0068** |
| | M: 0.273 ± 0.16 | M: 0.798 ± 0.22 | <0.0001**** |
| Ca, mg/dL | F: 12.73 ± 0.84 | F: 12.25 ± 0.69 | 0.2402^{ns} |
| | M: 12.67 ± 1.22 | M: 12.64 ± 0.81 | 0.9499^{ns} |
| P, mg/dL | F: 11.24 ± 2.45 | F: 7.14 ± 3.87 | 0.0394* |
| | M: 8.47 ± 3.35 | M: 7.92 ± 2.59 | 0.6993^{ns} |
| BUN, mg/dL | F: 47.11 ± 7.98 | F: 49.74 ± 7.89 | 0.5385^{ns} |
| | M: 54.35 ± 5.69 | M: 50.26 ± 6.11 | 0.1645^{ns} |

Statistical analysis was performed by Unpaired t test.
*P < 0.05.
**P < 0.01.
****P < 0.0001.
^{ns}P > 0.05—Not significant.
Comparison: WT vs. HPP prior to the treatment.
F—females; M—males.

After 60 days of administering AAV8-TNAP-D₁₀, serum ALP activity was significantly higher in female and male adult HPP mice and in WT mice compared to the vehicle (AAV8-GFP) group (FIG. 12C-12D, Table 5). Plasma PPi concentrations were higher in both female and male adult HPP mice, when compared to WT in the control AAV8-GFP treated mice. Plasma PPi levels were significantly lower in male adult HPP AAV8-TNAP-D₁₀ treated mice, but not in female AAV8-TNAP-D₁₀ treated mice, compared to AAV8-GFP treated adult HPP mice (FIG. 12E-12F, Table 5). AAV8-TNAP-D₁₀ treatment increased the calcium levels in WT females but not males. The calcium levels of adult HPP mice were not affected by the TNAP treatment (FIG. 12G-12H, Table 5). High phosphorus levels were observed in adult HPP mice treated with vehicle or with AAV8-TNAP-D₁₀ (FIG. 12I-12J, Table 5). There were no significant differences in BUN from serum samples of HPP and WT mice treated with AAV8-TNAP-D₁₀ and AAV8-GFP (FIG. 12K-12L, Table 5).

TABLE 5
Biochemical analysis of late-onset HPP and WT treated mice.

| | Genotype and treatment | | | | |
|---|---|---|---|---|---|
| Biochemical | WT | WT | HPP | HPP | ANOVA |
| markers | AAV8-GFP | AAV8-TNAP-D₁₀ | AAV8-GFP | AAV8-TNAP-D₁₀ | P-value |
| ALP, ng/mL | F: 44.67 ± 7.37 | F: 2482.00 ± 1030.00^{a} | F: 24.97 ± 4.62 | F: 214260.00 ± 23307.00 ^{b} | <0.0001 |
| | M: 31.53 ± 1.55 | M: 469950.00 ± 52957^{a} | M: 20.91 ± 2.62 | M: 484693.00 ± 155911.00 ^{bd} | <0.0001 |
| PPᵢ, μM | F: 0.243 ± 0.05 | F: 0.104 ± 0.05 | F: 0.752 ± 0.10^{c} | F: 0.530 ± 0.14^{d} | <0.001 |
| | M: 0.171 ± 0.10 | M: 0.072 ± 0.03 | M: 0.534 ± 0.13^{c} | M: 0.113 ± 0.05 ^{b} | <0.001 |
| Ca, mg/dL | F: 11.30 ± 1.08 | F: 14.02 ± 1.94^{a} | F: 11.52 ± 0.39 | F: 11.42 ± 0.47^{d} | <0.05 |
| | M: 11.34 ± 2.00 | M: 12.46 ± 0.57 | M: 12.85 ± 1.70 | M: 12.39 ± 0.75 | ns |
| P, mg/dL | F: 4.57 ± 0.61 | F: 5.73 ± 0.97 | F: 12.22 ± 0.89^{c} | F: 11.56 ± 2.72^{d} | <0.01 |
| | M: 3.85 ± 1.05 | M: 5.12 ± 1.18 | M: 8.47 ± 1.84^{c} | M: 7.94 ± 1.37^{d} | <0.05 |
| BUN, mg/dL | F: 54.02 ± 9.69 | F: 59.23 ± 15.69 | F: 48.62 ± 4.37 | F: 54.69 ± 3.82 | ns |
| | M: 50.11 ± 5.58 | M: 60.63 ± 12.95 | M: 56.21 ± 9.32 | M: 53.64 ± 5.14 | ns |

Statistical analysis was performed by One-way ANOVA followed by Tukey's multiple comparison test.
* P < 0.05.
** P < 0.01.
*** P < 0.001.
**** P < 0.0001.
^{ns}P > 0.05 - Not significant.
Comparisons: ^{a}WT: GFP vs. TNAP; ^{b} HPP: GFP vs. TNAP; ^{c}GFP: WT vs. HPP; ^{d}TNAP: WT vs. HPP.
F—females; M—males.

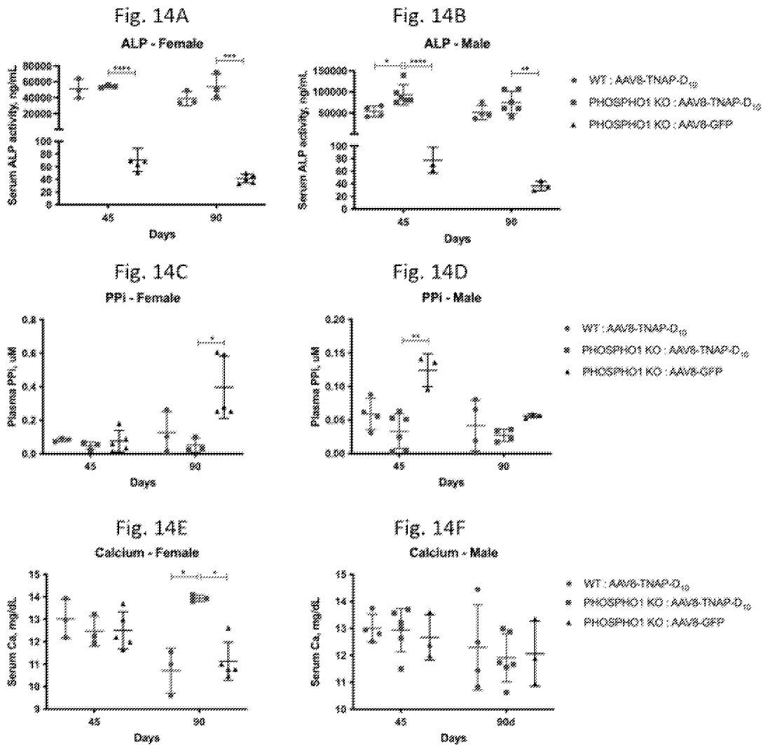

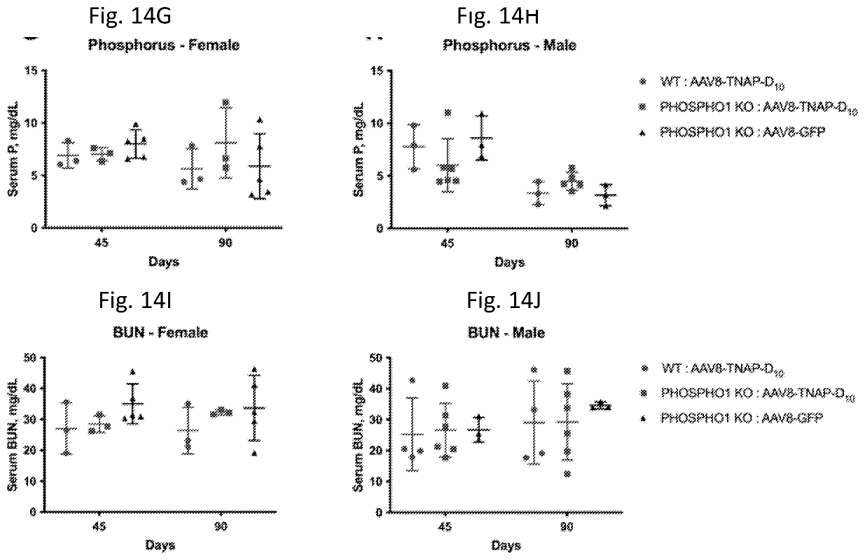

Biochemical markers were assessed in Phospho1−/− mice 45 and 90 days after AAV8-TNAP-D₁₀ treatment. Serum ALP activity was significantly higher and plasma PPi levels were lower in both female and male AAV8-TNAP-D₁₀ treated mice compared to AAV8-GFP treated mice (FIG. 14A-14D, Table 6). The levels of calcium in female but not male Phospho1−/− mice were increased after 90 days of AAV8-TNAP-D₁₀ injection when compared to the control AAV8-GFP treatment and AAV8-TNAP-D₁₀ treated WT mice (FIG. 14E-14F). No significant differences in phosphorus or BUN levels were observed after AAV8-TNAP-D₁₀ treatment (FIG. 14G-14J)

TABLE 6
Biochemical analysis of Phospho1 KO and WT treated mice

| | Genotype and Treatment | | | |
|---|---|---|---|---|
| Biochemical | WT | PHOSPHO1 KO | PHOSPHO1 KO | ANOVA |
| markers | AAV8-TNAP-D₁₀ | AAV8-TNAP-D₁₀ | AAV8-GFP | P-value |
| ALP, ng/mL | 45 d-F: 51122 ± 12212 | 45 d-F: 54101 ± 1807 ^{a} | 45 d-F: 70.65 ± 18.28 | <0.0001 |
| | 90 d-F: 38762 ± 8833 | 90 d-F: 54027 ± 16032^{a} | 90 d-F: 41.17 ± 6.973 | <0.0001 |
| | 45 d-M: 53781 ± 12749 | 45 d-M: 93250 ± 24000^{ab} | 45 d-M: 77.55 ± 20.72 | <0.05 |
| | 90 d-M: 51046 ± 17115 | 90 d-M: 74912 ± 26568 | 90 d-M: 36.56 ± 7.307 | <0.01 |
| PPᵢ, μM | 45 d-F: 0.08267 ± 0.01168 | 45 d-F: 0.048 ± 0.02234 | 45 d-F: 0.0756 ± 0.06438 | ns |
| | 90 d-F: 0.1273 ± 0.124 | 90 d-F: 0.05167 ± 0.04193^{a} | 90 d-F: 0.3964 ± 0.1853 | <0.05 |
| | 45 d-M: 53781 ± 12749 | 45 d-M: 93250 ± 24000^{ab} | 45 d-M: 77.55 ± 20.72 | <0.001 |
| | 90 d-M: 51046 ± 17115 | 90 d-M: 74912 ± 26568 | 90 d-M: 36.56 ± 7.307 | |
| Ca, mg/dL | 45 d-F: 13.02 ± 0.8675 | 45 d-F: 12.47 ± 0.6672 | 45 d-F: 12.51 ± 0.828 | ns |
| | 90 d-F: 10.71 ± 1.001 | 90 d-F: 13.94 ± 0.1407^{ab} | 90 d-F: 11.13 ± 0.8477 | <0.05 |
| | 45 d-M: 13.01 ± 0.5232 | 45 d-M: 12.94 ± 0.8042 | 45 d-M: 12.67 ± 0.8406 | ns |
| | 90 d-M: 12.3 ± 1.584 | 90 d-M: 11.91 ± 0.889 | 90 d-M: 12.06 ± 1.207 | |
| P, mg/dL | 45 d-F: 6.914 ± 1.209 | 45 d-F: 7.027 ± 0.6286 | 45 d-F: 8 ± 1.354 | ns |
| | 90 d-F: 5.631 ± 1.916 | 90 d-F: 8.115 ± 3.359 | 90 d-F: 5.878 ± 3.089 | |
| | 45 d-M: 7.77 ± 2.098 | 45 d-M: 6.014 ± 2.522 | 45 d-M: 8.595 ± 2.103 | ns |
| | 90 d-M: 3.356 ± 1.082 | 90 d-M: 4.486 ± 0.8501 | 90 d-M: 3.162 ± 1 | |
| BUN, mg/dL | 45 d-F: 27.02 ± 8.279 | 45 d-F: 28.49 ± 2.701 | 45 d-F: 35.01 ± 6.459 | ns |
| | 90 d-F: 26.39 ± 7.572 | 90 d-F: 32.3 ± 0.7988 | 90 d-F: 33.71 ± 10.56 | |
| | 45 d-M: 25.24 ± 11.73 | 45 d-M: 26.58 ± 8.674 | 45 d-M: 26.7 ± 3.987 | ns |
| | 90 d-M: 29.05 ± 13.36 | 90 d-M: 29.25 ± 12.31 | 90 d-M: 34.6 ± 1.126 | |

Improvements in the Skeleton with AAV8-TNAP-D10 Treatment without Evidence of Ectopic Calcification in Soft Organs

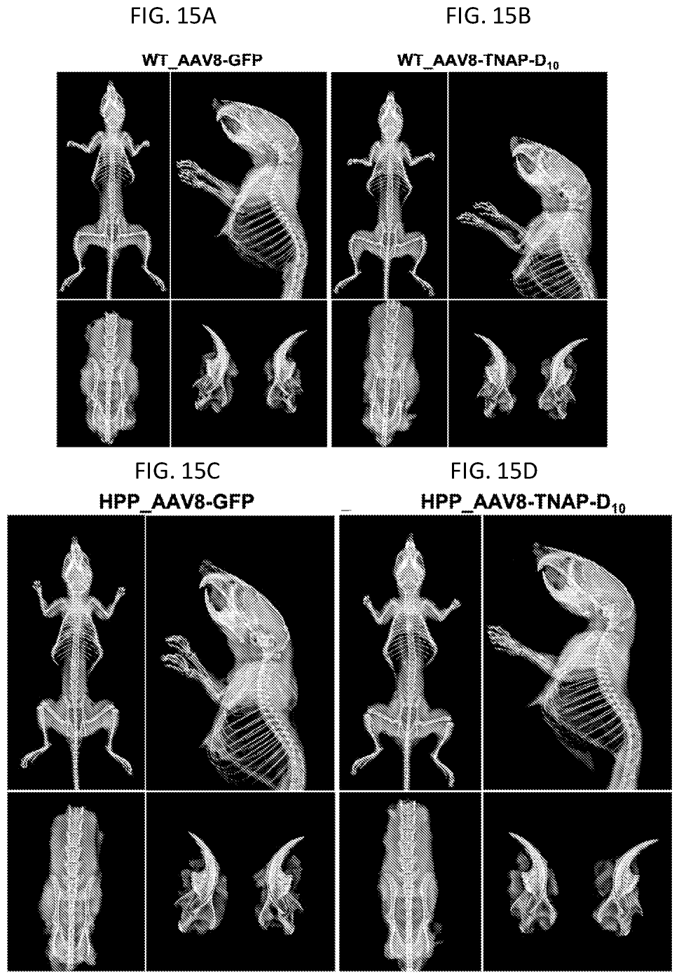

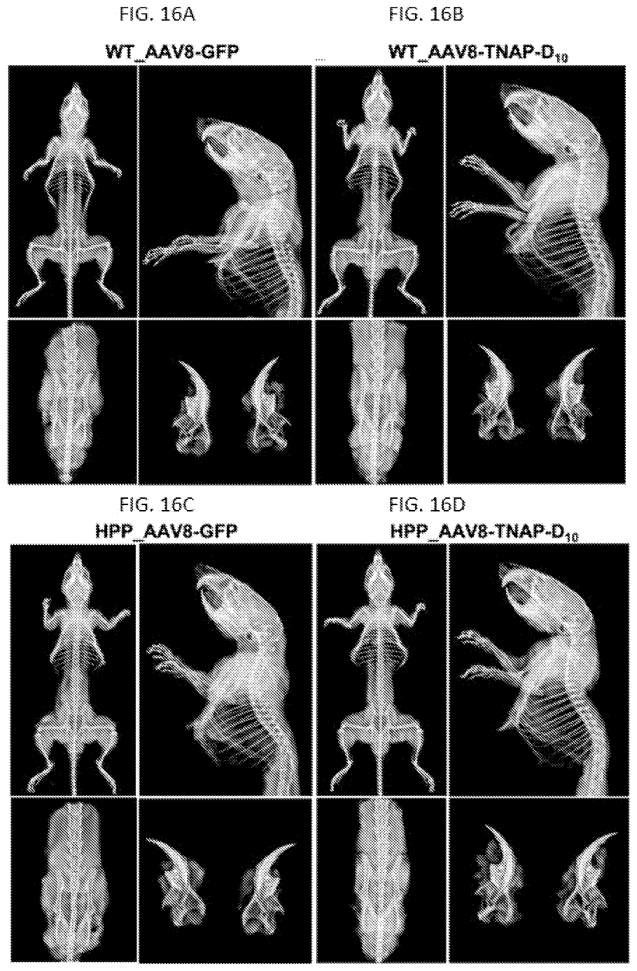

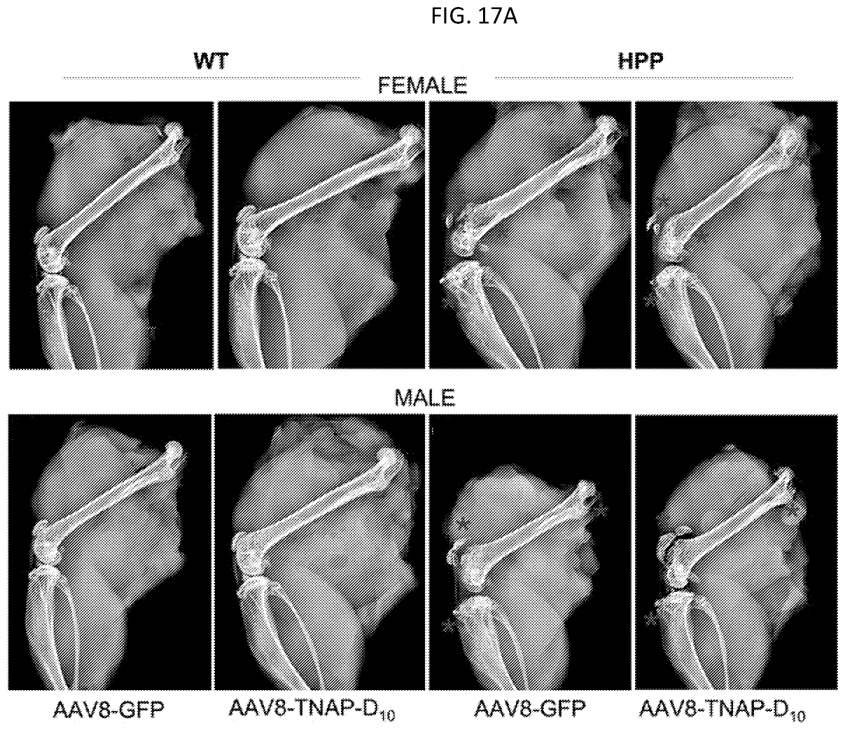

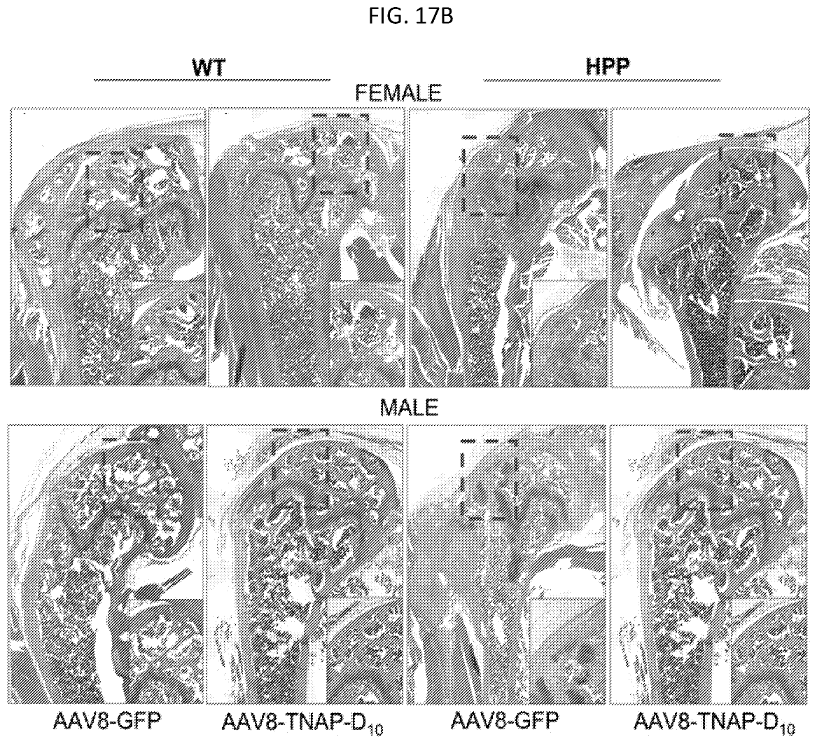

Radiographic images of female and male adult HPP mice revealed normal skeletal development when compared to WT littermates (FIG. 15A-15D; FIG. 16A-16D). However, long bones of female and male adult HPP mice presented defects in the epiphyseal and metaphyseal regions of the femur and tibia along with the patellar joint surface when compared to WT mice (FIG. 17A). Long bones of AAV8-TNAP-D₁₀ treated adult HPP mice were partially rescued as seen by the improvement of the proximal tibia and distal femur regions when compared to the AAV8-GFP treated adult HPP mice (FIG. 17B). In addition, there were no changes in the bone phenotype in AAV8-TNAP-D₁₀ treated WT mice when compared to AAV8-GFP treated WT mice (FIG. 17A). Histological sections of femurs stained with H&E from female and male adult HPP mice confirmed the damaged area visualized through X-ray images when compared to WT mice. A disorganized growth plate with a modified chondrocytes columnar organization was observed in adult HPP mice but not in WT mice (FIG. 17B). Femurs of AAV8-TNAP-D₁₀ treated adult HPP mice presented preserved growth plate area as indicated by the bone morphological organization related to the AAV8-GFP treated HPP mice (FIG. 17B). In addition, no histological changes in the bone phenotype in AAV8-TNAP-D₁₀ treated WT mice were observed when compared to WT mice treated with AAV8-GFP (FIG. 17B).

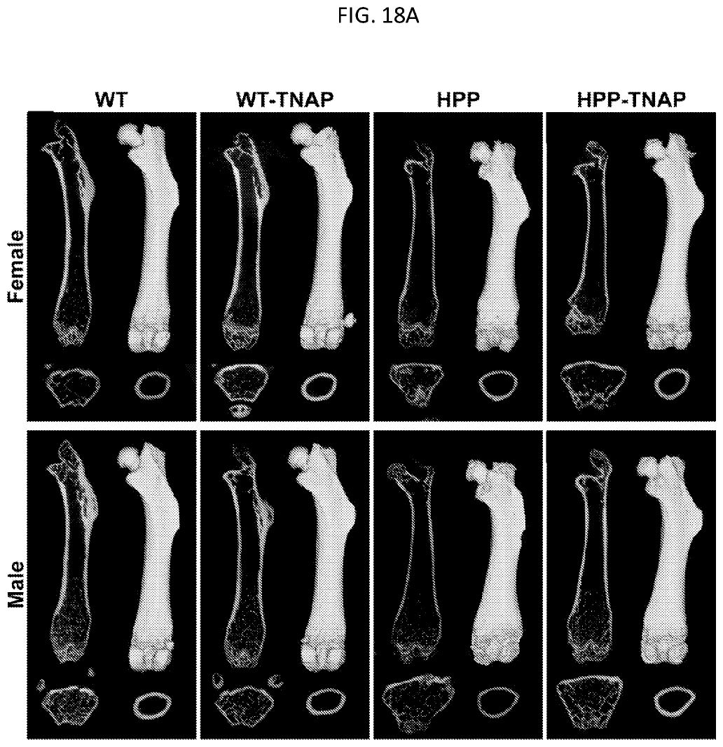

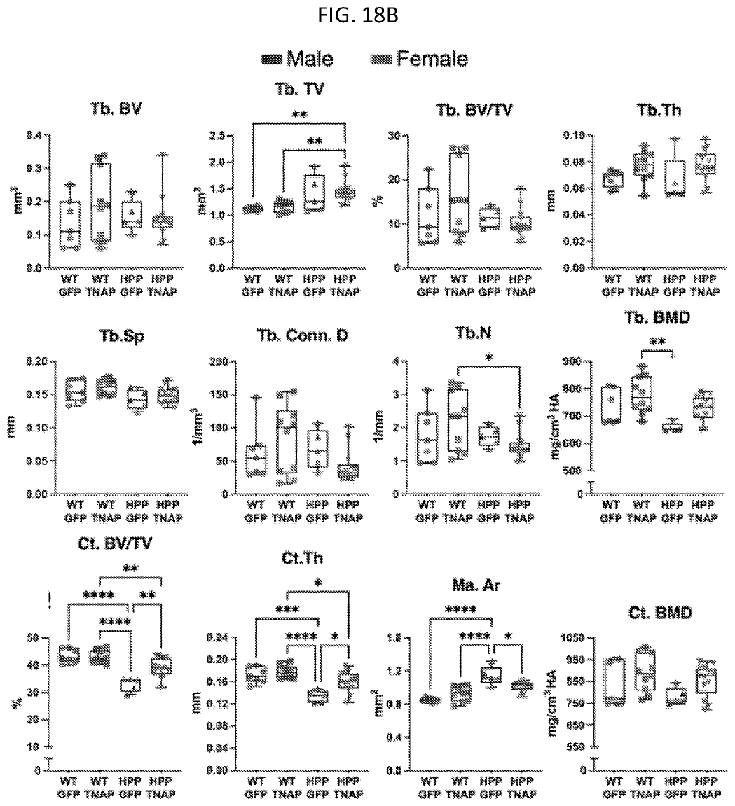

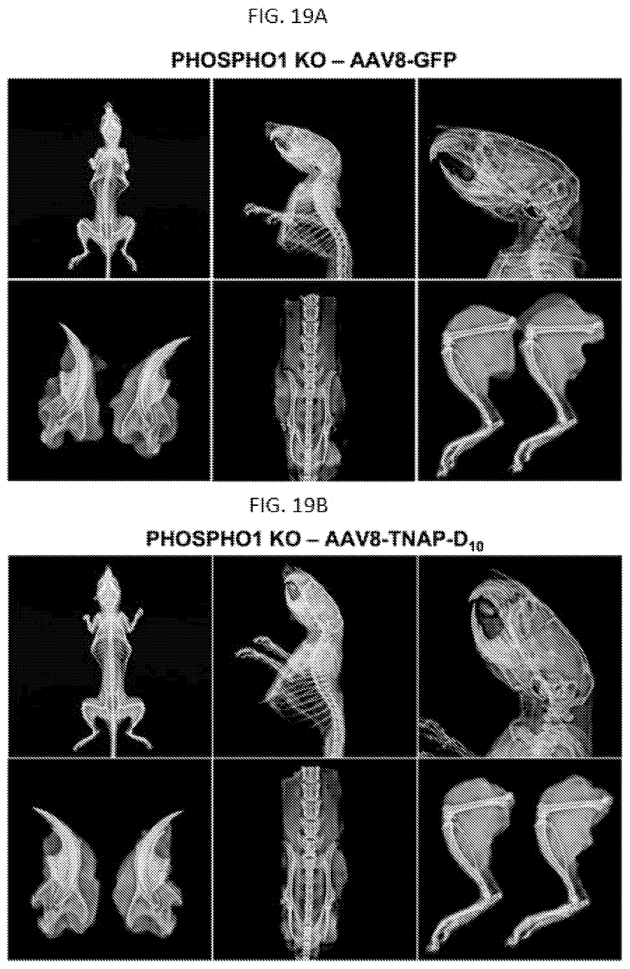

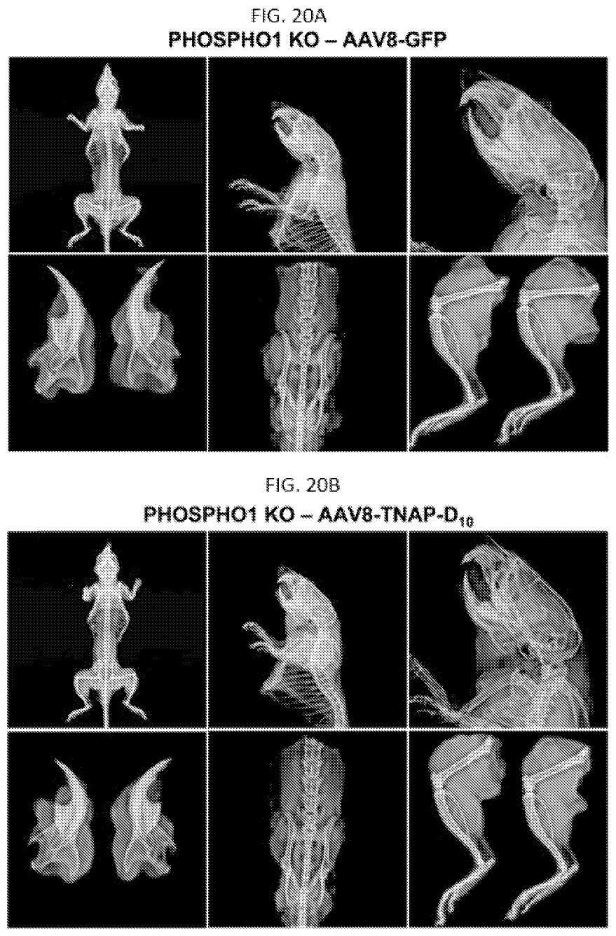

Micro-CT analysis of bone parameters showed that untreated HPP mice had a statistically significant reduction in cortical bone volume fraction (Ct. BV/TV) compared with untreated and AAV8-TNAP-D₁₀ treated WT littermates. The reduced cortical BV/TV in untreated HPP mice was associated with a significantly reduced cortical thickness (Ct. Th) and increased marrow area (Ma. Ar) vs. untreated and treated WT littermates. However, the cortical bone mineral density (Ct. BMD) of untreated HPP mice was not statistically significant different from treated and untreated WT littermates. Gene therapy using the AAV8-TNAP-D₁₀ vector significantly improved cortical bone in HPP mice, including bone fraction (Ct. BV/TV), thickness, and marrow area. Notably, no statistically significant difference was observed between treated and untreated WT mice, suggesting no adverse effects of AAV8-TNAP-D₁₀ vector-mediated gene therapy over 60 days (FIG. 18A-18B).

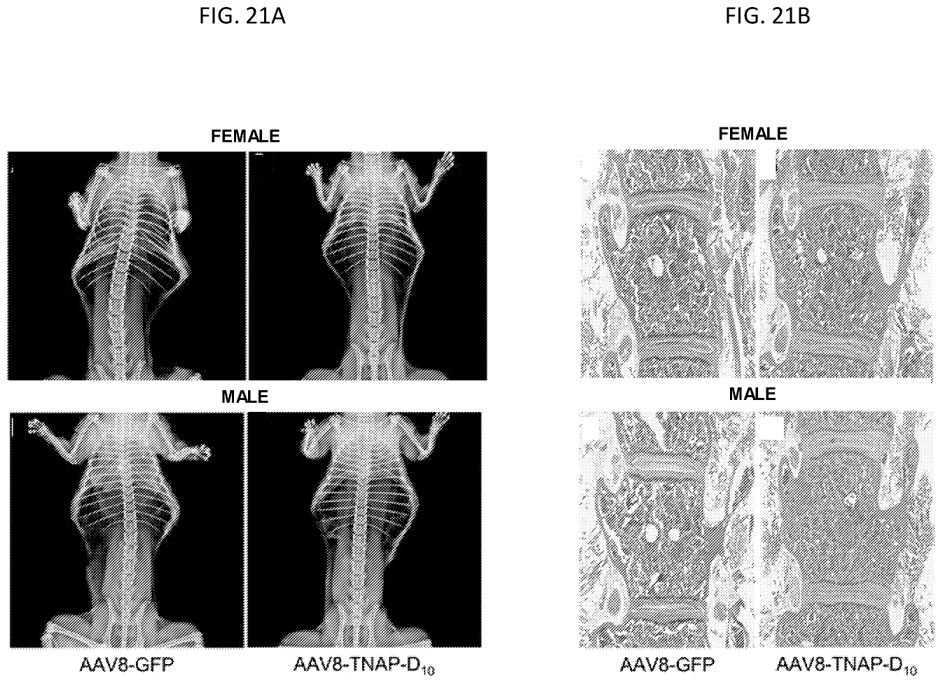

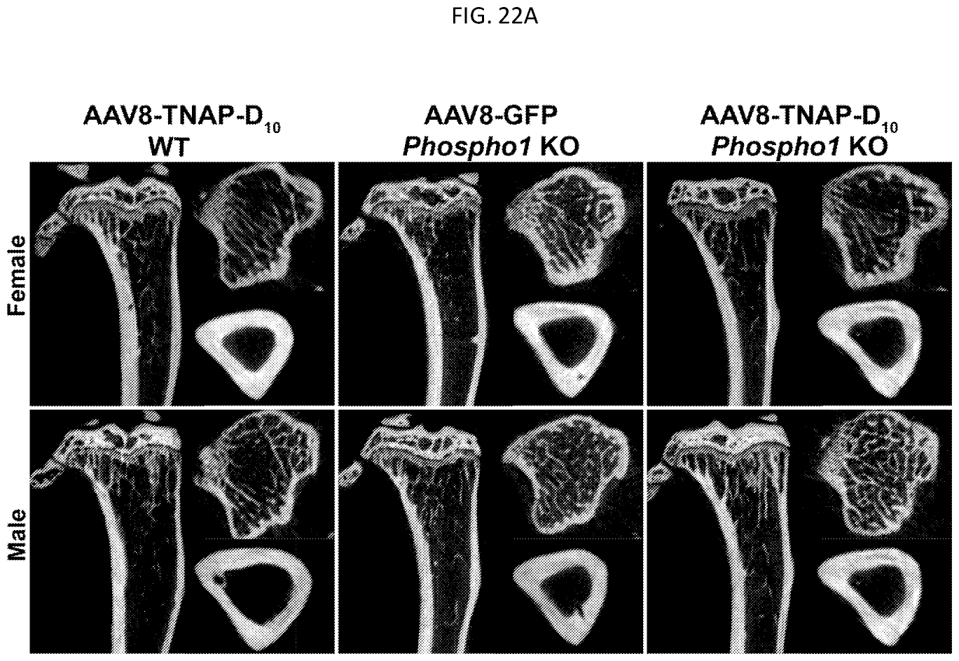

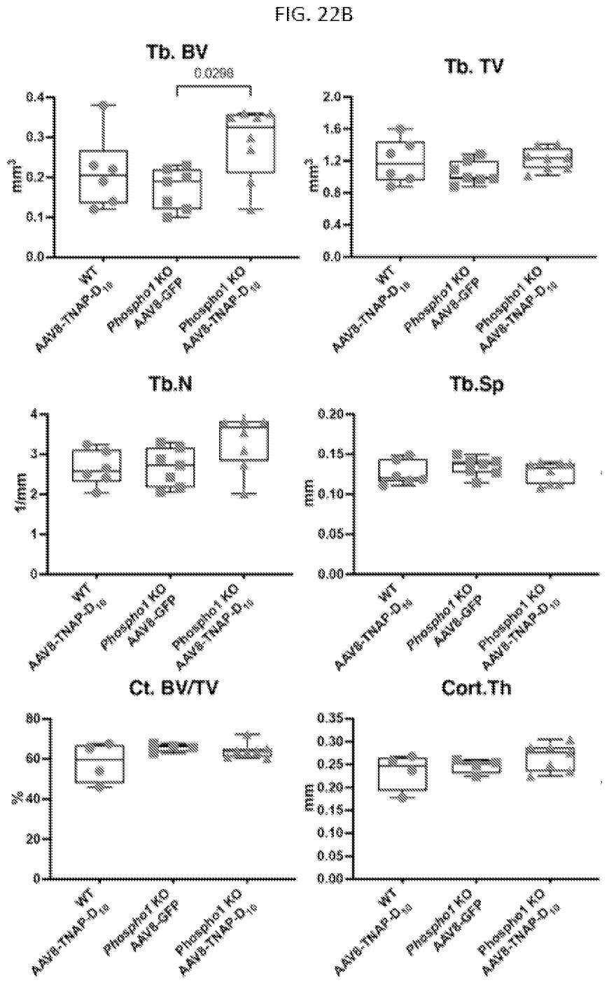

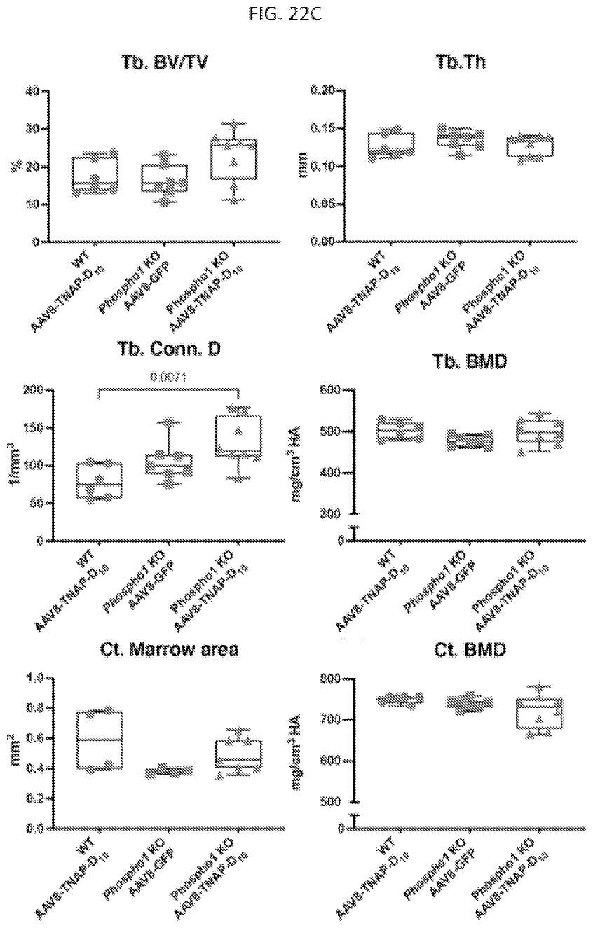

Radiographic images of female and male Phospho1−/− mice showed apparent normal skeletal development, except for a spinal deformity, i.e., the abnormal moderate and severe degrees of scoliosis in vertebrae of untreated Phospho1−/− mice. (FIG. 19A-19B; FIG. 20A-20B). Interestingly, a single dose of AAV8-TNAP-D₁₀ improved the scoliotic curves and corrected this congenital manifestation in female and male Phospho1−/− mice as seen 90 days after injection (FIG. 21A). H&E staining of the vertebrae showed more trabecular bone and a reduced marrow area in AAV8-TNAP-D₁₀ treated mice when compared to AAV8-GFP control (FIG. 21B). Micro-CT analysis of tibiae in AAV8-TNAP-D₁₀ Phospho1−/− treated mice showed increased trabecular bone volume (Tb. BV) and trabecular connectivity density (Tb. Conn. D) when compared to AAV8-GFP Phospho1−/− treated mice and AAV8-TNAP-D₁₀ WT mice, respectively (FIGS. 22A-22C)

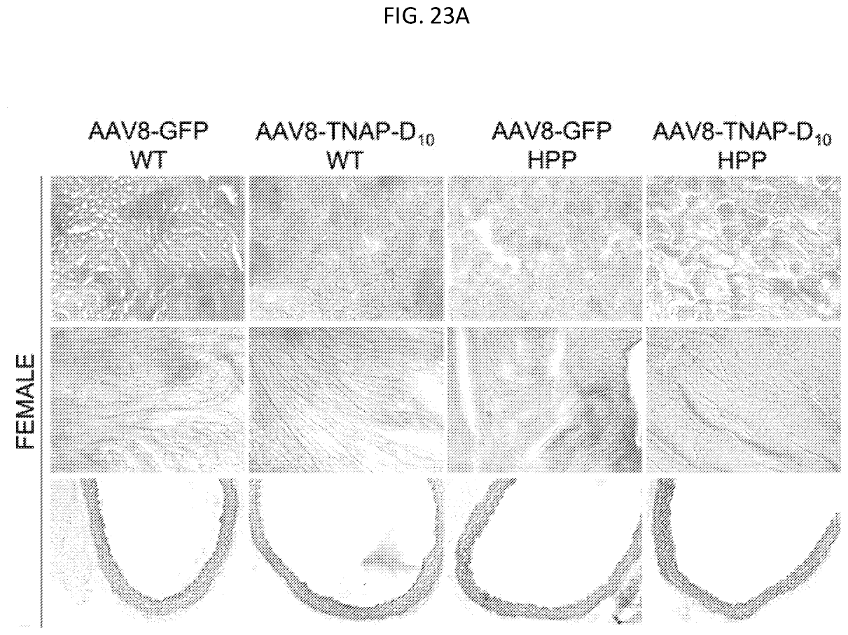

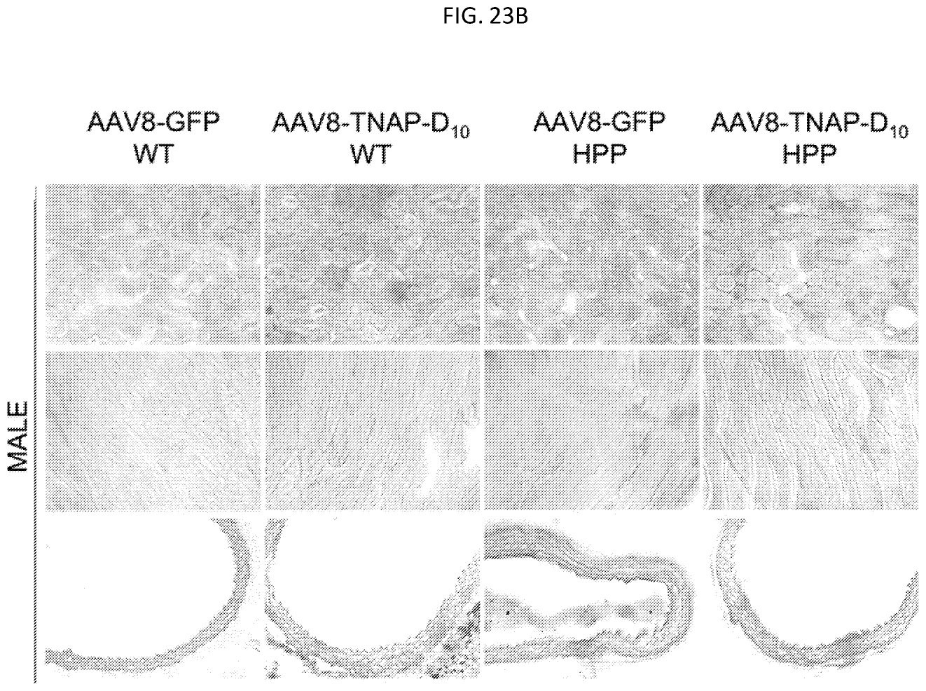

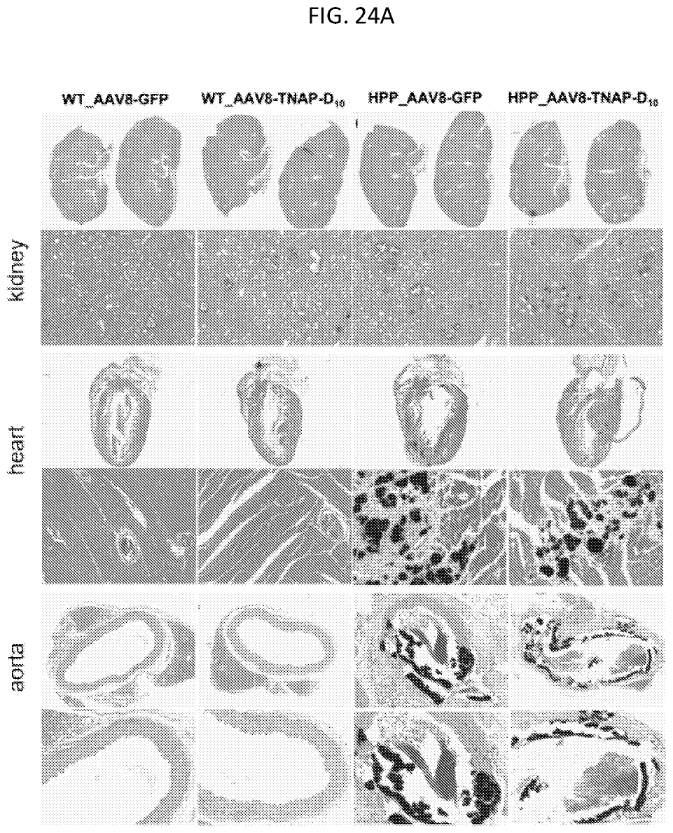

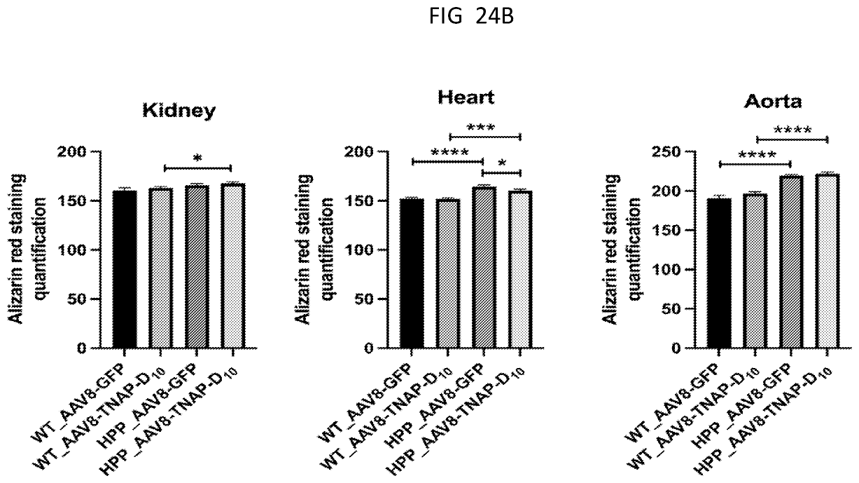

In addition, the treatment with the AAV8-TNAP-D₁₀ vector did not promote ectopic calcification in the kidney, heart, or aorta of female and male adult HPP mouse model (FIG. 23A-23B) in this short treatment window (60 days of treatment), and it did not increase the calcification of soft organs in adult HPP when chronic kidney disease was superimposed as comorbidity after 60 days of treatment (FIGS. 24A-24M).

### Characterization of Dentoalveolar Complex in Both Osteomalacia Models

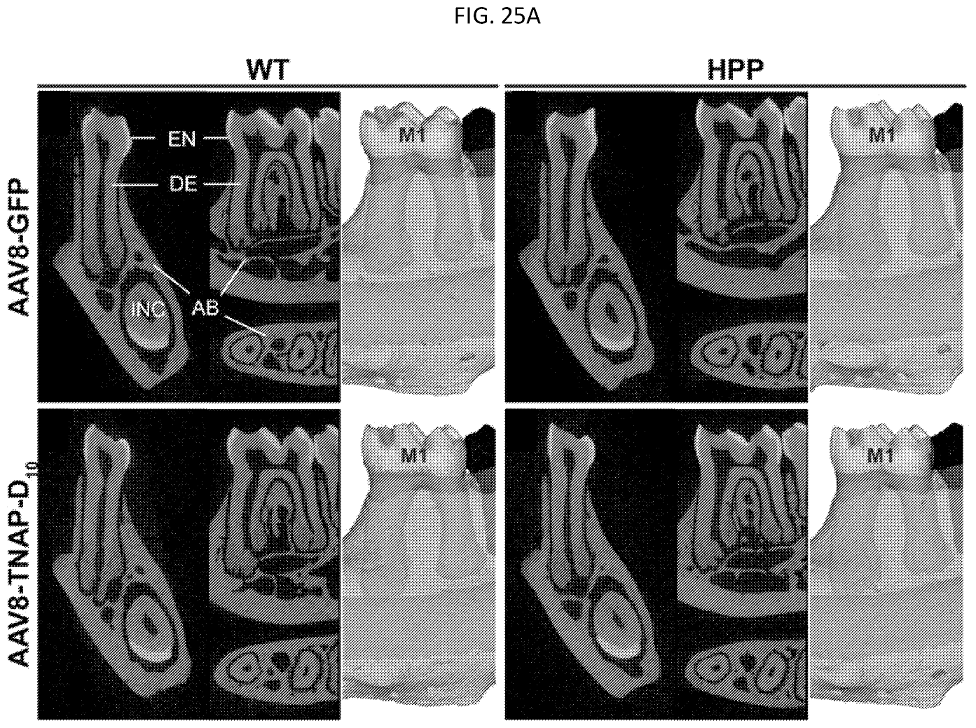

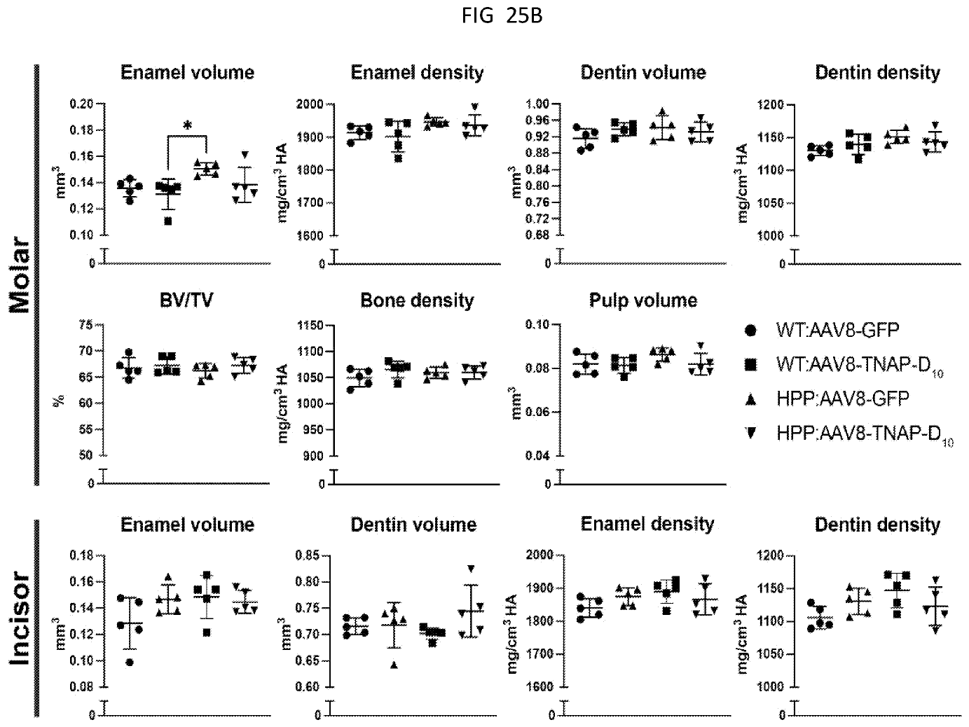

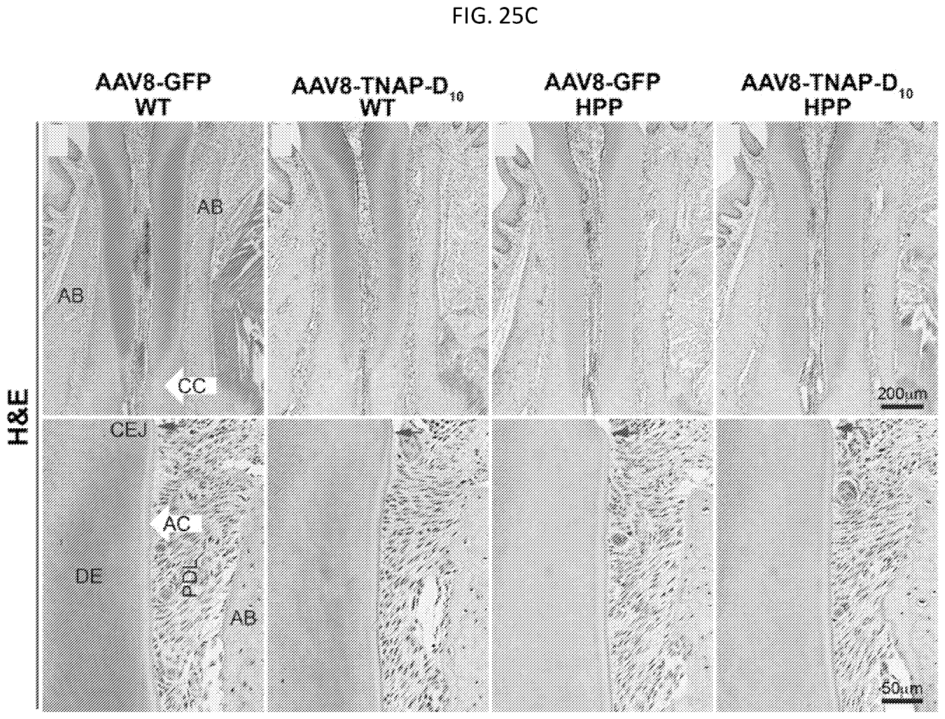

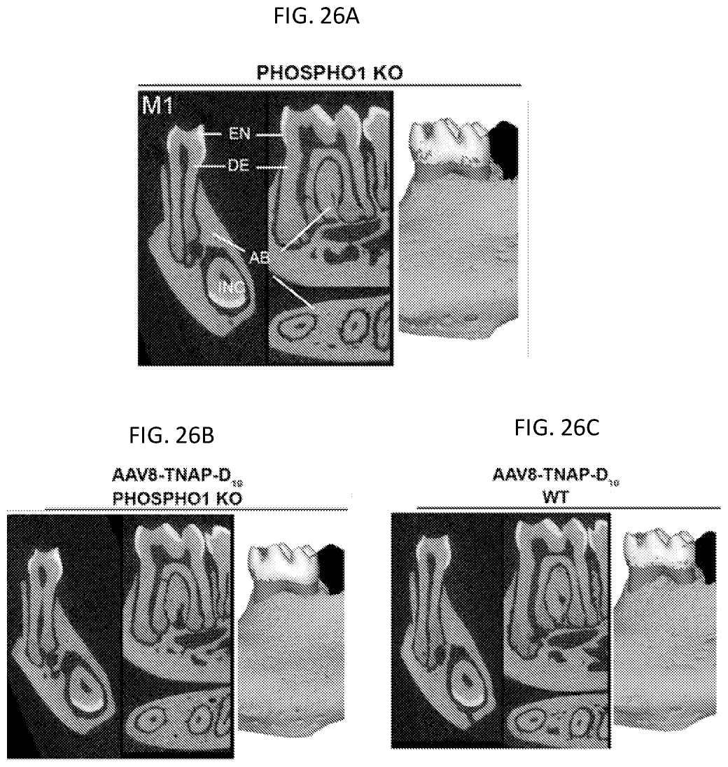

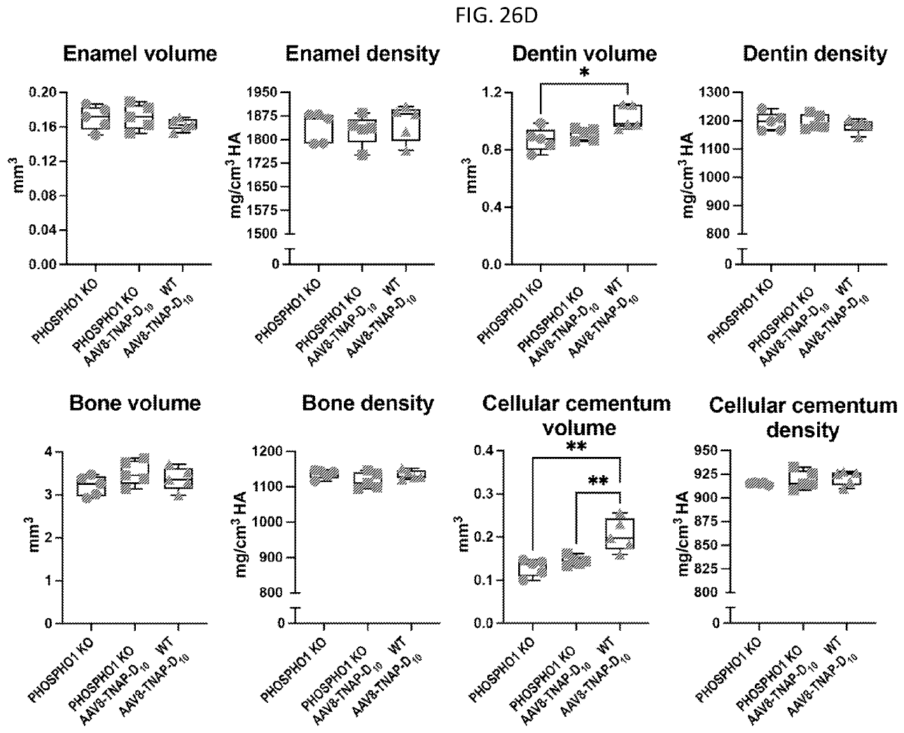

Micro-CT analysis of the first mandibular molar and associated alveolar bone showed that adult HPP mice had no evident defects in enamel, dentin/cementum, bone volumes, and mineral densities, compared with untreated WT or AAV8-TNAP-D₁₀ treated WT and HPP mice. However, there was a statistically significant difference in the enamel volume between AAV8-TNAP-D₁₀ treated WT and untreated HPP. Similarly, micro-CT analysis of continuously erupting incisors showed no significant difference in enamel and dentin volumes and densities. Together, these results suggest that Alpl ablation in Prx1-expressing dental mesenchymal cells may be dispensable for dentoalveolar formation and mineralization. Additionally, AAV8-TNAP-D₁₀-mediated gene therapy for 60 days had no adverse effects as no difference was observed between treated and untreated WT teeth. Histological analysis of untreated HPP teeth revealed no morphological differences compared with untreated WT or both AAV8-treated-HPP and WT littermates. No obvious difference in tissue organization, periodontal attachments, or acellular cementum between treated and untreated HPP (FIG. 25A-25C). Similar results were found in the dentoalveolar complex of Phospho1−/− mice, showing milder mineralization defects. No evident phenotype changes except for an increase in the cellular cementum volume in AAV8-TNAP-D₁₀ treated WT mice when compared to Phospho1−/− treated and untreated mice (FIG. 26).

### Sequences

| SEQ | | |
|---|---|---|
| ID | | |
| NO | Description | Sequence |
| 15 | TNAP | MISPFLVLAIGTCLTNSLVPEKEKDPKYWRDQAQETLKYALELQK |
| | | LNTNVAKNVIMFLGDGMGVSTVTAARILKGQLHHNPGEETRLE |
| | | MDKFPFVALSKTYNTNAQVPDSAGTATAYLCGVKANEGTVGVS |
| | | AATERSRCNTTQGNEVTSILRWAKDAGKSVGIVTTTRVNHATPS |
| | | AAYAHSADRDWYSDNEMPPEALSQGCKDIAYQLMHNIRDIDVIM |
| | | GGGRKYMYPKNKTDVEYESDEKARGTRLDGLDLVDTWKSFKPR |
| | | YKHSHFIWNRTELLTLDPHNVDYLLGLFEPGDMQYELNRNNVTD |
| | | PSLSEMVVVAIQILRKNPKGFFLLVEGGRIDHGHHEGKAKQALHE |
| | | AVEMDRAIGQAGSLTSSEDTLTVVTADHSHVFTFGGYTPRGNSIF |
| | | GLAPMLSDTDKKPFTAILYGNGPGYKVVGGERENVSMVDYAHN |
| | | NYQAQSAVPLRHETHGGEDVAVFSKGPMAHLLHGVHEQNYVPH |
| | | VMAYAACIGANLGHCAPASSAGSLAAGPLLLALALYPLSVLF |
| 16 | CAG | GCGTTACATAACTTACGGTAAATGGCCCGCCTGGCTGACCGCC |
| | promoter | CAACGACCCCCGCCCATTGACGTCAATAATGACGTATGTTCCC |
| | | ATAGTAACGCCAATAGGGACTTTCCATTGACGTCAATGGGTGG |
| | | AGTATTTACGGTAAACTGCCCACTTGGCAGTACATCAAGTGTA |
| | | TCATATGCCAAGTACGCCCCCTATTGACGTCAATGACGGTAAA |
| | | TGGCCCGCCTGGCATTATGCCCAGTACATGACCTTATGGGACT |
| | | TTCCTACTTGGCAGTACATCTACGTATTAGTCATCGCTATTACC |
| | | ATGGTCGAGGTGAGCCCCACGTTCTGCTTCACTCTCCCCATCTC |
| | | CCCCCCCTCCCCACCCCCAATTTTGTATTTATTTATTTTTTAATT |
| | | ATTTTGTGCAGCGATGGGGGCGGGGGGGGGGGGGGGGCGCGC |
| | | GCCAGGCGGGGCGGGGCGGGGCGAGGGGCGGGGCGGGGCGA |
| | | GGCGGAGAGGTGCGGCGGCAGCCAATCAGAGCGGCGCGCTCC |
| | | GAAAGTTTCCTTTTATGGCGAGGCGGCGGCGGCGGCGGCCCTA |
| | | TAAAAAGCGAAGCGCGCGGGGGCG |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating a subject with a soft bone disease comprising: administering a viral vector comprising a mineral-targeted alkaline phosphatase under the control of a tissue non-specific promotor to the subject in an intramuscular injection to a muscle, wherein administering the viral vector treats the soft bone disease, wherein the mineral-targeted alkaline phosphatase comprises tissue non-specific alkaline phosphatase (TNAP); and wherein the soft bone disease is caused by PHOSPHO1 deficiency.

2. The method of claim 1, wherein the tissue non-specific promotor comprises a CAG promotor.

3. The method of claim 1, wherein the viral vector comprises an adeno-associated vector.

4. The method of claim 1, wherein the viral vector comprises an adeno-associated virus type 8 (AAV8) vector.

5. (canceled)

6. The method of claim 1, wherein the mineral-targeted alkaline phosphatase further comprises a bone targeting sequence linked to the C-terminus of TNAP.

7. The method of claim 6, wherein the bone targeting sequence is a deca-aspartate (D₁₀) sequence.

8. The method of claim 1, wherein the soft bone disease is hypophosphatasia (HPP).

9. The method of claim 1, wherein the soft bone disease is hypophosphatasia (HPP), and wherein the hypophosphatasia is pediatric hypophosphatasia or infantile hypophosphatasia.

10. The method of claim 1, wherein the soft bone disease is hypophosphatasia (HPP), and wherein the hypophosphatasia is late-onset hypophosphatasia.

11. (canceled)

12. The method of claim 1, wherein the subject is a human.

13. (canceled)

14. (canceled)

15. The method of claim 1, wherein administering the viral vector results in increased plasma alkaline phosphatase (ALP) activity for at least two months.

16. (canceled)

17. (canceled)

18. The method of claim 1, wherein following administering the viral vector, the viral vector does not result in oncogenic effect in the subject.

19. The method of claim 1, wherein the viral vector does not diffuse from the muscle.

20.-33. (canceled)

34. A method of treating a subject with a dental disorder comprising: administering a viral vector comprising a mineral-targeted alkaline phosphatase under the control of a tissue non-specific promotor to the subject in an intramuscular injection, wherein administering the viral vector treats the dental disorder; and wherein the mineral-targeted alkaline phosphatase comprises tissue non-specific alkaline phosphatase (TNAP).

35. The method of claim 34, wherein the tissue non-specific promotor comprises a CAG promotor.

36. The method of claim 34, wherein the viral vector comprises an adenoviral-associated virus.

37. The method of claim 36, wherein the viral vector comprises an adeno-associated virus type 8 (AAV8) vector.

38. (canceled)

39. The method of claim 34, wherein the mineral-targeted alkaline phosphatase further comprises a sequence for bone targeting linked to the C-terminus of TNAP.

40. The method of claim 39, wherein the bone targeting sequence is a deca-aspartate (D₁₀) sequence.

41. The method of claim 34, wherein the dental disorder comprises at least one of a dentoalveolar disorder, teeth hypomineralization, and a periodontal disorder.
